(19) United States
(12) Patent Application Publication
(10) Pub. No.: US 2024/0173072 A1
(43) Pub. Date: May 30, 2024

(54) DEVICES AND METHODS FOR TRANSCATHETER HEART VALVE LEAFLET CUTTING AND MODIFICATION

(71) Applicants: Vivek Rajagopal, Atlanta, GA (US); Jaime Eduardo Sarabia, Mableton, GA (US); Yenchin Liao, Cary, NC (US); Alfred Raschdorf, St. James, NY (US)

(72) Inventors: Vivek Rajagopal, Atlanta, GA (US); Jaime Eduardo Sarabia, Mableton, GA (US); Yenchin Liao, Cary, NC (US); Alfred Raschdorf, St. James, NY (US)

(21) Appl. No.: 18/523,047

(22) Filed: Nov. 29, 2023

Related U.S. Application Data
(60) Provisional application No. 63/385,253, filed on Nov. 29, 2022.

(51) Int. Cl. A61B 18/14

(52) U.S. Cl. CPC A61B 18/1492; A61B 2018/00369

(57) ABSTRACT

A minimally invasive and steerable leaflet grasper for intracardiac implantation into a heart chamber for grasping a heart leaflet and modifying the leaflet. The leaflet grasper includes a proximal control knob for controlling the distal grasping end of the leaflet grasper having a grasping arm, gripper, and laceration element for securing the leaflet and modifying the leaflet. The method includes using a single leaflet grasper to secure and lacerate the leaflet.

### CROSS REFERENCE TO RELATED APPLICATIONS

The present application claims the benefit of and priority to Provisional Patent Application Ser. No. 63/385,253 filed Nov. 29, 2022, the disclosure of which is herein incorporated by reference.

### FIELD OF INVENTION

The present invention relates generally to medical devices and systems for cutting heart valve leaflets and methods of use of these devices and systems. More specifically, the invention pertains to medical devices and systems which are inserted minimally invasively to cut or modify any heart valve leaflet of the heart or any valve leaflet of any prosthetic heart valve, using one or more leaflet cutting and modification devices and systems.

### BACKGROUND OF INVENTION

Transcatheter valve replacement is the minimally invasive implantation of a valve to replace a malfunctioning heart valve. Transcatheter aortic valve replacement (TAVR) replaces a malfunctioning aortic valve, and transcatheter mitral valve replacement (TMVR) replaces a malfunctioning mitral valve. Although TAVR and TMVR can be quite effective in treating a broad range of valvular pathologies, these devices can adversely affect native anatomy in some patients. For example, in some patients TAVR can push a native aortic leaflet against a coronary ostium, causing coronary artery obstruction. Similarly, in some patients TMVR can push the anterior mitral leaflet into the left ventricular outflow tract (LVOT), causing LVOT obstruction.

Several investigators have developed techniques to prevent these complications in susceptible patients. To prevent coronary obstruction with TAVR, investigators have used existing guiding catheters and wires to lacerate native leaflets prior to TAVR implantation. In BASILICA (Bioprosthetic or native Aortic Scallop Intentional Laceration to prevent Iatrogenic Coronary Artery obstruction), catheters and wires are used to lacerate one or more native aortic leaflets using radiofrequency energy. When a native aortic valve leaflet is cut from base to tip, it splays open when it is pushed against the aorta by the TAVR. Thus, blood can flow freely through the TAVR frame and past the cut leaflet into the coronary artery. Similarly, in LAMPOON (Laceration of the Anterior Mitral leaflet to Prevent Outflow ObtructioN), catheters and wires are used to lacerate the anterior mitral leaflet. The split anterior leaflet also splays open when pushed by the TMVR, allowing blood to flow through the TMVR frame and freely into the LVOT.

The LAMPOON technique can also be used in patients with prior transcatheter mitral or tricuspid valve edge-to-edge therapy to allow for TMVR or transcatheter tricuspid valve replacement (TTVR). Transcatheter edge-to-edge repair (TEER) is a minimally invasive mitral or tricuspid valve repair whereby a device is used to approximate the anterior and posterior mitral valve leaflets together thereby reducing mitral or tricuspid valve regurgitation. For example, MitraClip® (Abbott Vascular, Santa Clara, California, USA) is commercially approved for minimally invasive mitral valve repair; the PASCAL repair system (Edwards LifeSciences, Irvine, California, USA) and other devices are being evaluated in clinical trials for minimally invasive mitral valve repair. These devices are also being evaluated in clinical trials for minimally invasive tricuspid valve repair.

In some patients, however, implantation of these devices does not reduce valvular regurgitation sufficiently, or valvular regurgitation recurs at some point after implantation. In these circumstances, the devices can be surgically excised via open heart surgery with subsequent repair or replacement of the valve. Given that most of these patients are not good surgical candidates, an alternative strategy is to use the LAMPOON technique to cut one of the mitral or tricuspid leaflets next to the device, thereby restoring the full opening of the native valve, which is necessary for the implantation and normal functioning of either a TMVR or TTVR.

Nonetheless, using LAMPOON, either to re-open a valve after TEER, or to prevent LVOT obstruction from TMVR, is technically complicated and requires specialized expertise to be feasible. Therefore, many patients who might benefit from these techniques, might not have access to the therapy. Similarly, BASILICA also requires a high level of expertise, which prevents many patients from having access to its benefits. Furthermore, even with a high level of expertise, there is a need for easier, more reproducible, and more robust devices and methods to facilitate leaflet modification prior to transcatheter therapies. In particular, some patients might require more extensive cutting or resection, such as patients undergoing TAVR in TAVR procedures, where standard BASILICA might be insufficient to prevent coronary obstruction.

### SUMMARY OF INVENTION

Presented herein are medical devices and systems which are inserted minimally invasively onto or around any heart valve to allow cutting and/or resection of any portion of the heart valve leaflet. The steerable leaflet grasper can function to grab any heart valve leaflet, lacerate any portion of it, and can also resect any portion of it. The steerable leaflet catheter can encircle any leaflet next to an edge-to-edge device, allowing laceration of the leaflet to free the device from one of the leaflets, restoring normal valve excursion to allow implantation of either a transcatheter mitral or tricuspid valve replacement.

In one aspect, the steerable leaflet grasper and steerable leaflet catheter each comprise a proximal control handle and shaft with a deflectable distal end. The deflectable distal end of the steerable leaflet grasper connects to a grasper end. The deflectable distal end of the leaflet catheter comprises a lumen to pass a laceration element. The end of the steerable leaflet grasper and steerable leaflet catheter may be advanced through the intracardiac chambers over a guidewire, or may advance without a guidewire.

The proximal control handle of the leaflet grasper and leaflet catheter comprises levers, knobs, buttons, or rotary controls that affect deflection of distal end. The proximal control handle of the leaflet grasper also has controls that affect closing/opening of grasper arm, actuation of the gripper, and activation of the laceration element. The shaft of the leaflet grasper and leaflet catheter comprises control and guidewire lumens, and the distal end of the shaft is deflectable by the deflection control of the proximal control handle. The distal end of the leaflet catheter comprises a lumen which allows passage of a cutting element that encircles valve tissues and goes back up another lumen in the shaft of the leaflet catheter. The distal end of the leaflet grasper shaft connects to a grasper end, which comprises a grasping arm, gripper element, and laceration element.

The grasping arm is of any width, length, or thickness, and is composed of any combination of plastic polymers or metallic alloys. The grasping arm, via any type of hinge or articulating mechanism, may extend at any variable angle or distance from the shaft when controlled by the closing/opening control on the proximal control handle. The gripper arm is also of any shape or size, and composed of any combination of plastic polymers. The gripper arm may or may not have any number of protrusions on its surface, composed of any plastic polymer or metallic alloy. Via the gripper control on the proximal control handle, the gripper arm may extend to any variable angle or distance such that it may sandwitch leaflet tissue between itself and the grasper arm. The laceration element may be any type of barb, hook, blade, needle, or protrusion, composed of any type of plastic polymer or metallic alloy. Via activation by the laceration control of the proximal control handle, the laceration element engages the leaflet tissue and may be fixed in position relative to either the grasper or gripper arms. When laceration element is fixed relative to the grasper or gripper arms, movement of either the grasper or gripper arms or both cuts the leaflet tissue. Alternatively, the laceration element may move in any vertical, horizontal, polygonal, circular, elliptical, or sinusoidal path relative to the grasper or gripper arms, thereby cutting leaflet tissue.

In another aspect, the laceration element is a wire, of any diameter that is electroconductive, that is integrated to either the gripper or grasper arms, or is connected to the catheter shaft. The wire runs through the catheter shaft through a port of the proximal control handle, which allows external connection of the wire to a generator that provides alternating current in the radiofrequency range. When current is applied to the wire, it can cut the leaflet along the path of the wire.

In another aspect, once the leaflet is cut, the gripper arm may retract back to the catheter shaft and the grasper arm may extend away from the gripper arm, releasing the cut leaflet tissue. Conversely, the gripper and grasper arms may remain closed on the tissue, and retraction of the catheter shaft pulls the cut tissue away from the rest of the leaflet, allowing resection of a portion of leaflet tissue.

In another aspect, the leaflet catheter cuts leaflet tissue away from an edge-to-edge implant, without resecting tissue. Using the deflection control of the proximal control handle, the leaflet catheter advances (either over a guidewire or without a guidewire) through one of the orifices of the valve created by the edge-to-edge implant. Once next to the implant, the deflection control bends the distal tip around the leaflet tissue until the distal tip makes contact with an opening of a more proximal portion of the catheter shaft, which connects to a separate lumen in the catheter shaft. From the proximal control handle, an electroconductive wire is passed into a port of either of the lumens of the catheter shaft, until it exits out the second lumen and out a second port of the proximal control handle. The wire is connected to an electrical current generator; once the portion of the wire next to leaflet tissue is exposed, either by retraction of the proximal catheter shaft relative to the distal shaft or by straightening the distal shaft away from the proximal shaft, application of current to the wire with retraction of the wire cuts the leaflet tissue way from the edge-to-edge device.

Related methods of operation are also provided. Other apparatuses, methods, systems, features, and advantages of the medical devices and systems that are implanted minimally invasively in the heart will be or become apparent to one with skill in the art upon examination of the following Figures and detailed description. It is intended that all such additional apparatuses, methods, systems, features, and advantages be included within this description, be within the scope of the medical devices and systems that are implanted minimally invasively in the heart and be protected by the accompanying claims.

### BRIEF DESCRIPTION OF THE FIGURES

FIG. 1 is a cut-away perspective view of a heart;

FIG. 2 is a cut-away perspective view of an aortic root;

FIG. 3 is a cut-away perspective view of a heart showing a transcatheter heart valve positioned across the mitral valve in the heart with the anterior mitral leaflet displaced and causing left ventricular outflow tract obstruction;

FIG. 4 is a cut-away perspective view of a heart showing a transcatheter heart valve positioned across the mitral valve in the heart with the anterior mitral leaflet cut and resected, preventing left ventricular outflow tract obstruction, by the devices and methods according to the present invention;

FIG. 5 is a cut-away perspective of an aortic root showing a transcatheter heart valve positioned across the aortic valve with the aortic valve leaflet causing coronary artery obstruction;

FIG. 6 is a cut-away perspective of an aortic root showing a transcatheter heart valve positioned across the aortic valve with the aortic valve leaflet cut and resected, preventing coronary artery obstruction, by the devices and methods according to the present invention;

FIG. 7 is a cut-away perspective of a heart showing a transcatheter edge-to-edge device implanted on a mitral valve;

FIG. 8 is a cut-away perspective of a heart showing a transcatheter edge-to-edge device released from the anterior mitral valve leaflet by the devices and methods according to this invention;

FIG. 9 is a cut-away perspective of a heart showing a transcatheter edge-to-edge device implanted on a tricuspid valve;

FIG. 10 is a cut-away perspective of a heart showing a transcatheter edge-to-edge device released from a tricuspid valve leaflet by the devices and methods according to this invention;

FIG. 11A is a side-elevational view of a leaflet grasper according to this invention;

FIG. 11B is a magnified side-elevational view of the proximal control handle of a leaflet grasper according to this invention;

FIG. 12A is a side-elevational view of a leaflet grasper showing rotation of the deflection control according to this invention;

FIG. 12B is a side-elevational view of a leaflet grasper showing the shaft bent downwards;

FIG. 13 is a magnified side-elevational view of a proximal control handle;

FIG. 14 is a magnified side-elevational view of a leaflet grasper end with the grasper arm open;

FIG. 15 is a magnified side-elevational view of a proximal control handle with the leaflet grasper control in baseline state;

FIG. 16 is a magnified side-elevational view of a proximal control handle with the leaflet grasper control pressed inwards;

FIG. 17 is a magnified side-elevational view of a leaflet grasper end with the grasper arm open and gripper adjacent to shaft;

FIG. 18 is magnified side-elevational view of a leaflet grasper end with the grasper arm open and the gripper closed onto the grasper arm;

FIG. 19 is a magnified side-elevational view of a proximal control handle with the laceration engagement control in baseline state;

FIG. 20 is a magnified side-elevational view of a proximal control handle with the laceration engagement control pushed forward;

FIG. 21 a magnified side-elevational view of a leaflet grasper end with the gripper close to the shaft, with the laceration element within the grasper arm;

FIG. 22 is a magnified side-elevational view of a leaflet grasper end with the gripper close to the shaft, with the laceration element extended out of the grasper arm;

FIG. 23 is a magnified side-elevational view of a leaflet grasper end with the gripper close to the grasper arm, with the laceration element within the grasper arm;

FIG. 24 is FIG. 23 with the laceration element extended from the grasper arm;

FIG. 25 is a magnified side-elevational view of a leaflet grasper end with both the grasper arm and gripper close to the shaft, with the laceration element within the grasper arm;

FIG. 26. is FIG. 25. with the laceration element extended from the grasper arm;

FIG. 27 is a magnified side-elevational view of a leaflet grasper end with the grasper arm open, gripper near the shaft, with the laceration element within the gripper;

FIG. 28 is FIG. 27 with the laceration element extended from the gripper;

FIG. 29 is a magnified side-elevational view of a leaflet grasper end with the grasper arm open, gripper adjacent to grasper arm, with the laceration element within the gripper;

FIG. 30 is FIG. 29 with the laceration element extended from the gripper;

FIG. 31 is a magnified side-elevational view of a leaflet grasper end closed with the laceration element within the gripper;

FIG. 32 is a FIG. 31 with laceration element extended from the gripper;

FIG. 33 is a magnified side-elevational view of a leaflet grasper end that is closed with the laceration element within the shaft;

FIG. 34 is a FIG. 31 with laceration element extended from the shaft of the leaflet grasper;

FIG. 35 is a magnified side elevational view of a proximal control handle with the laceration movement control in its baseline position;

FIG. 36 is FIG. 35 with the laceration movement control rotated;

FIG. 37 is a magnified side elevational view of a leaflet grasper end with the laceration element at the top of the gripper;

FIG. 38 is FIG. 37 with the laceration element moved to the bottom of the gripper;

FIG. 39A is a side elevational view of a radiofrequency current generator connected to an electrosurgical wire running through the electrosurgical wire port of a leaflet grasper;

FIG. 39B is a magnified side elevational view of a proximal control handle of FIG. 39A;

FIG. 40A is a cut-away perspective of a heart with a leaflet grasper advancing over a guidewire attached to previously placed interventricular septal anchor;

FIG. 40B is a magnified view of FIG. 40A;

FIG. 41A is a cut-away perspective of a heart with a leaflet grasper advancing next to a guidewire attached to previously placed interventricular septal anchor;

FIG. 41B is a magnified view of FIG. 41A;

FIG. 42A is a cut-away perspective of a heart with a leaflet grasper advancing without any guidewire or anchor;

FIG. 42B is a magnified view of FIG. 42A;

FIG. 43A is a cut-away perspective of a heart with a leaflet grasper advanced over a guidewire attached to anchor with grasper arm opened;

FIG. 43B is a magnified view of FIG. 43A;

FIG. 44A is a cut-away perspective of a heart with a leaflet grasper advanced next to a guidewire attached to anchor with grasper arm opened;

FIG. 44B is a magnified view of FIG. 44A;

FIG. 45A is a cut-away perspective of a heart with a leaflet grasper with the grasper arm opened without any guidewire or anchor;

FIG. 45B is a magnified view of FIG. 45A;

FIG. 46A is a cut-away perspective of a heart with a leaflet grasper advanced over a guidewire attached to anchor with the gripper arm closed on a leaflet;

FIG. 46B is a magnified view of FIG. 46A;

FIG. 47A is a cut-away perspective of a heart with a leaflet grasper advanced next to a guidewire attached to anchor with gripper arm closed on a leaflet;

FIG. 47B is a magnified view of FIG. 47A;

FIG. 48A is a cut-away perspective of a heart with a leaflet grasper without any guidewire or anchor with a gripper arm closed on a leaflet;

FIG. 48B is a magnified view of FIG. 48A;

FIG. 49A is a cut-away perspective view of a heart with a leaflet grasper advanced over a guidewire attached to an anchor with the grasper end closed on a leaflet;

FIG. 49B is a magnified view of FIG. 49A;

FIG. 50A is a cut-away perspective view of a heart with a leaflet grasper advanced next to a guidewire attached to anchor with the grasper end closed on a leaflet;

FIG. 50B is a magnified view of FIG. 50A;

FIG. 51A is a cut-away perspective view of a heart with a leaflet grasper advanced without a guidewire or anchor with the grasper end closed on a leaflet;

FIG. 51B is a magnified view of FIG. 51A;

FIG. 52 is a cut-away perspective view of a heart with a leaflet grasper advanced from left atrium into left ventricle and across the aortic valve;

FIG. 53 is a cut-away perspective view of a heart with a leaflet grasper advanced over a wire from left atrium into left ventricle and across the aortic valve;

FIG. 54 is FIG. 52 with the leaflet grasper arm open and abutting the aortic valve leaflet;

FIG. 55 is FIG. 53 with the leaflet grasper arm open and abutting the aortic valve leaflet;

FIG. 56 is FIG. 54 with the leaflet gripper closed onto the aortic leaflet;

FIG. 57 is FIG. 55 with the leaflet gripper closed onto the aortic leaflet;

FIG. 58 is FIG. 56 with the leaflet grasper arm closed onto the aortic leaflet;

FIG. 59 is FIG. 57 with the leaflet grasper arm closed onto the aortic leaflet;

FIG. 60 is a side elevational view of a leaflet grasper with alternative grasper end;

FIG. 61 is FIG. 60 with grasper arm open;

FIG. 62 is FIG. 61 with gripper closed onto the grasper arm;

FIG. 63 is a cut-away perspective view of a heart with a leaflet grasper advanced retrograde from aorta across the aortic valve;

FIG. 64 is a cut-away perspective view of a heart with a leaflet grasper advanced retrograde over a wire from aorta across the aortic valve;

FIG. 65 is FIG. 60 with the leaflet grasper arm open and abutting the aortic valve leaflet;

FIG. 66 is FIG. 61 with the leaflet grasper arm open and abutting the aortic valve leaflet;

FIG. 67 is FIG. 62 with the leaflet gripper closed onto the aortic leaflet;

FIG. 68 is FIG. 63 with the leaflet gripper closed onto the aortic leaflet;

FIG. 69 is FIG. 64 with the leaflet grasper arm closed onto the aortic leaflet;

FIG. 70 is FIG. 65 with the leaflet grasper arm closed onto the aortic leaflet;

FIG. 71A is a side-elevational view of a leaflet splitter according to this invention;

FIG. 71B is a magnified side-elevational view of the proximal control handle of a leaflet splitter according to this invention;

FIG. 72A is a side-elevational view of a leaflet splitter showing rotation of the deflection control according to this invention;

FIG. 72B is a side-elevational view of a leaflet splitter showing the shaft bent downwards;

FIG. 73A is a side-elevational view of a leaflet splitter showing rotation of the tip deflection knob causing the tip to deflect 90 degrees inward;

FIG. 73B is a side-elevational view of a leaflet splitter showing further rotation of the tip deflection knob causing the tip to deflect 180 degrees inward;

FIG. 73C is a side-elevational view of a leaflet splitter showing further rotation of the tip deflection knob causing the tip to deflect enough to mate with the telescoping port;

FIG. 74A is a side-elevational view of a leaflet splitter showing the telescoping catheter control being pushed;

FIG. 74B is a magnified side-elevational view of a proximal control handle of a leaflet splitter showing the telescoping catheter control being pushed;

FIG. 75A is a side-elevational view of a leaflet splitter showing the telescoping catheter control pushed forward with the telescoping catheter advancing outside the telescoping catheter port;

FIG. 75B is a magnified side-elevational view of a proximal control handle of a leaflet splitter showing the telescoping catheter control pushed forward;

FIG. 76A is a side-elevational view of a leaflet splitter showing the telescoping catheter control completely pushed forward with the telescoping catheter completely advanced outside the telescoping catheter port;

FIG. 76B is a magnified side-elevational view of a proximal control handle of a leaflet splitter showing the telescoping catheter control completely pushed forward;

FIG. 77A-C is a side-elevational view of a leaflet splitter with the telescoping catheter bent toward the tip of the leaflet splitter;

FIG. 78 is a cut-away perspective view of the heart with a transcatheter edge-to-edge device on the mitral valve with a guidewire attached to an interventricular septal anchor;

FIG. 79 is FIG. 78 with a leaflet splitter advancing over the guidewire;

FIG. 80A is a cut-away perspective of the heart with the leaflet splitter tip encircling a mitral leaflet;

FIG. 80B is a magnified view of FIG. 80A;

FIG. 81 is FIG. 80A with the leaflet splitter being pulled up;

FIG. 82 is a magnified view of the leaflet splitter with an electrosurgical wire going through the leaflet splitter and wrapped around a mitral leaflet;

FIG. 83 is a magnified view of the leaflet splitter after it has cut a mitral leaflet away from the transcatheter edge-to-edge device;

FIG. 84 is a magnified view of the leaflet splitter with an electrosurgical wire after it has cut a mitral leaflet away from the transcatheter edge-to-edge device;

FIG. 85 is a magnified view of a leaflet splitter with the telescoping catheter advanced around a leaflet;

FIG. 86 is a magnified view of a leaflet splitter with the telescoping catheter and electrosurgical wire around a leaflet;

FIG. 87 is a magnified view of a leaflet splitter with the telescoping catheter and electrosurgical wire after it has cut a leaflet away from a transcatheter edge-to-edge device;

FIG. 88 is a cut-away perspective of a heart with a leaflet grasper advancing over a guidewire that is not attached an interventricular septal anchor;

FIG. 89 is a cut-away perspective of a heart with a leaflet grasper advanced over a guidewire not attached to anchor with grasper arm opened;

FIG. 90 is a cut-away perspective of a heart with a leaflet grasper advanced over a guidewire not attached to anchor with the gripper arm closed on a leaflet;

FIG. 91 is a cut-away perspective view of a heart with a leaflet grasper advanced over a guidewire not attached to an anchor with the grasper end closed on a leaflet;

FIG. 92 is a cut-away perspective of a heart with a leaflet grasper advancing across an aortic valve over a guidewire that is not attached an interventricular septal anchor;

FIG. 93 is a cut-away perspective of a heart with a leaflet grasper advanced across an aortic valve over a guidewire not attached to anchor with grasper arm opened;

FIG. 94 is a cut-away perspective of a heart with a leaflet grasper advanced across an aortic valve over a guidewire not attached to anchor with the gripper arm closed on a leaflet;

FIG. 95 is a cut-away perspective view of a heart with a leaflet grasper advanced across an aortic valve over a guidewire not attached to an anchor with the grasper end closed on a leaflet;

### DETAILED DESCRIPTION OF THE INVENTION

The present invention is understood more readily by reference to the following detailed description, examples, and claims, and their previous and following description. Before the present system, devices, and/or methods are disclosed and described, it is to be understood that this invention is not limited to the specific systems, devices, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. Those skilled in the relevant art will recognize that many changes can be made to the aspects described, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used herein, the singular forms “a,” “an” and “the” include plural referents unless the context clearly dictates otherwise. Thus, for example, reference to a “tether” includes aspects having two or more tethers unless the context clearly indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not. For the purposes of describing and defining the present invention it is noted that the use of relative terms, such as “substantially”, “generally”, “approximately”, and the like, are utilized herein to represent an inherent degree of uncertainty that is attributed to any quantitative comparison, value, measurement, or other representation. These terms are also utilized herein to represent the degree by which a quantitative representation may vary from a stated reference without resulting in a change in the basic function of the subject matter at issue.

The application relates to medical devices and systems to be minimally invasively inserted in the heart 1, and methods of use of these devices and systems. The medical devices and systems used to modify heart valve leaflets is a minimally invasive leaflet assembly for intracardia implantation into a heart chamber for grasping a heart leaflet and modifying the leaflet. The assembly includes a leaflet grasper 10 and a leaflet catheter 20 which, according to one aspect of the invention are both steerable. The leaflet grasper 10 may be used to cut or cut/resect any portion of any leaflet, such as the anterior mitral leaflet 7, posterior mitral leaflet 8, any one of the three tricuspid leaflets 14, any of the three aortic valve leaflets 17, any one of the leaflets 102 of a prosthetic mitral or tricuspid valve, or any one of the leaflets 202 of a prosthetic aortic valve. The steerable leaflet catheter 20 may used to encircle and cut away any portion of the leaflets of a mitral valve 6 or of a tricuspid valve 13 from an edge-to-edge device 301.

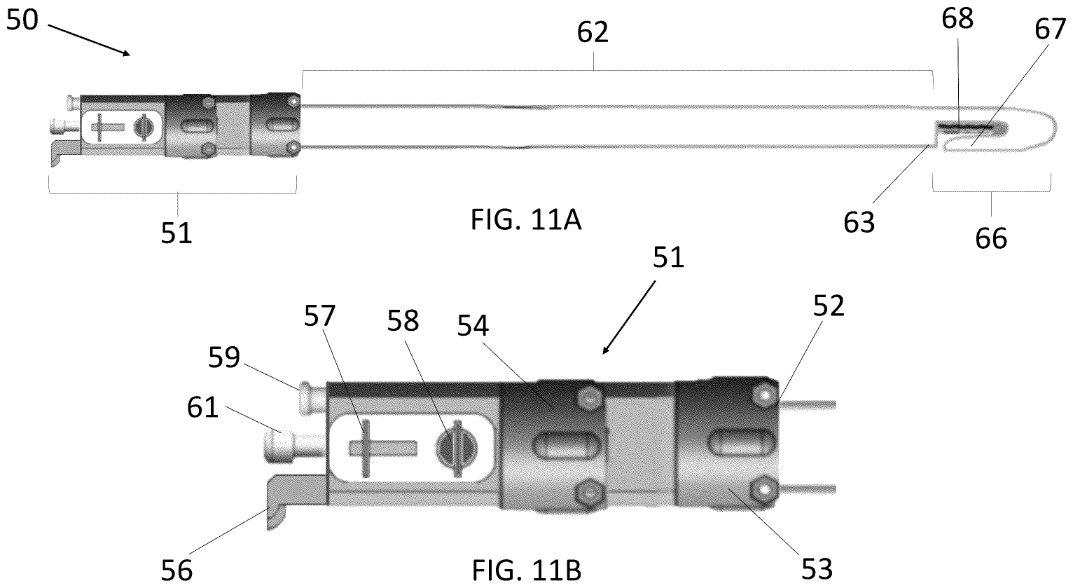

### The Steerable Leaflet Grasper [FIGS. 11A-B, 12-38]

Referring to FIG. 11A-B, the leaflet grasper 50 consists of a proximal control handle 51, catheter shaft 62, and grasper end 66. Any portion of the leaflet grasper may be composed of flexible or inflexible materials, including, but not limited to, polyether block amide (PEBA or PEBAX), polyetheretherketone (“PEEK”), polytetrafluoroethylene (“PTFE”), thermoplastic polyurethane (“TPU”), other polymers, stainless steel, cobalt chromium, nickel, titanium, or any combination thereof.

In one aspect, the proximal control handle 51, fused to the proximal portion 52 of the catheter shaft 62, has a deflection knob 53, which controls the deflection of the catheter shaft 62, grasper control 54, which opens and closes the grasper arm 67 of the grasping end 66, gripper control 56, which lowers or raises the gripper 68, laceration engagement control 57, which engages or disengages laceration element 69, laceration movement control 58, which moves laceration element 69, electrosurgical wire port 59, and guidewire port 61.

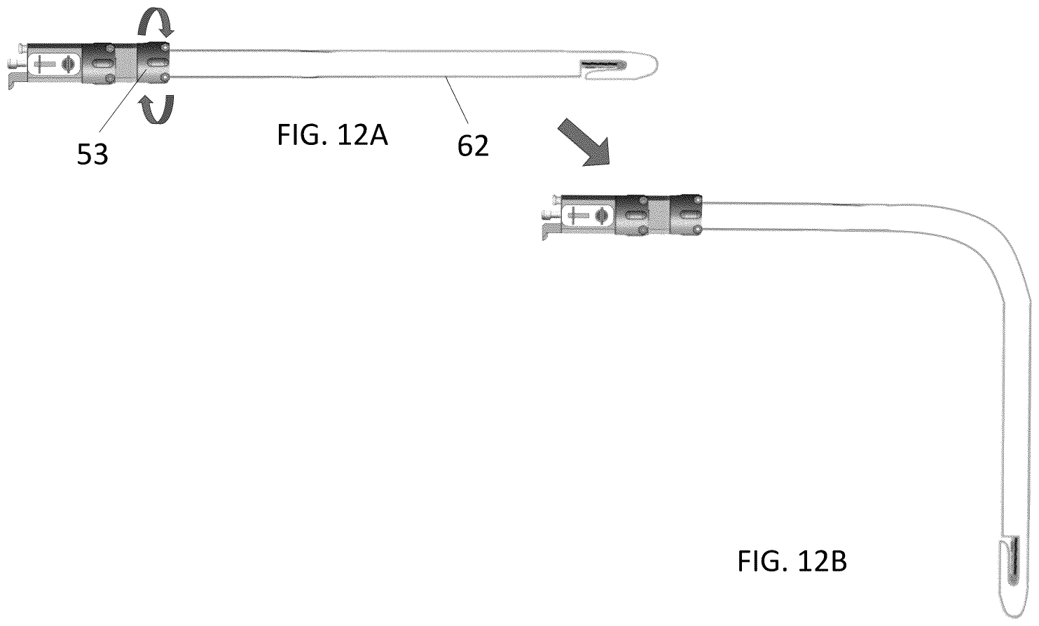

Referring to FIGS. 12A-B, the shaft 62 can be changed from a straight configuration to a bent configuration and back by rotation of the deflection knob 53 of the proximal control handle 51. Rotation of the deflection knob 53 in one direction bends the catheter shaft 62. This bending may be in one or more locations along the length of the shaft, and the one or more locations may bend to any desirable angle so that the shaft may be bent into any type of curve. Rotation of the deflection in the opposite direction reverses the one or more bends along one or more locations of the shaft 62.

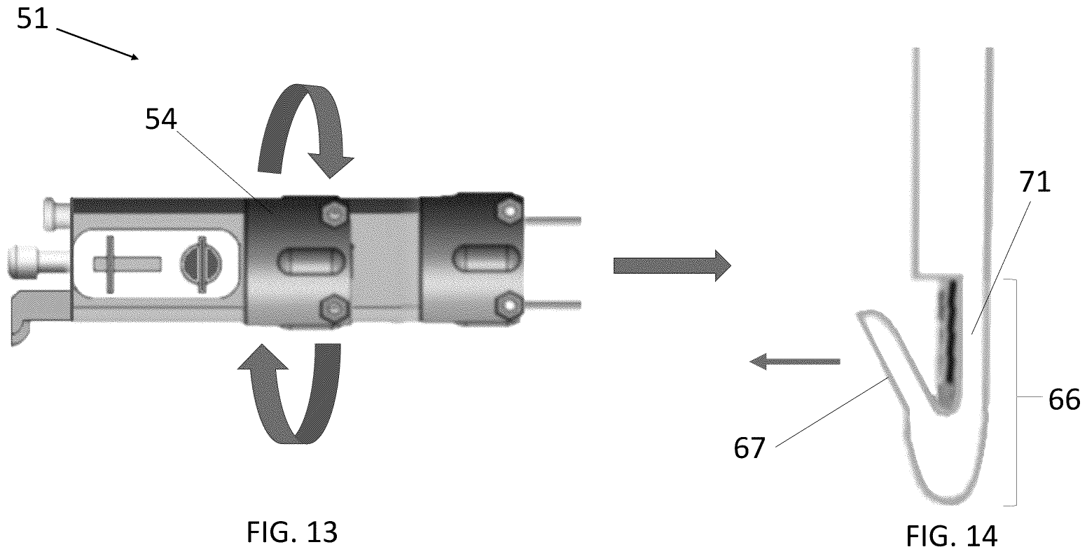

Referring to FIG. 13-14, rotation of the grasper control 54 of the proximal control handle 51 extends the grasper arm 67 outward from the shaft 71 of the grasper end 66. Reverse rotation of the grasper control 54 moves the grasper arm 67 back to the shaft 71 of the grasper end 66. Grasper arm 67 may extend outward at any angle via anytime of spring or hinge element connecting it to grasper end 66. The grasper arm 67 may be any length, width, or thickness. Grasper arm 67 may take any polygonal, circular, or elliptical shape, and may be composed of any combination of materials outlined above.

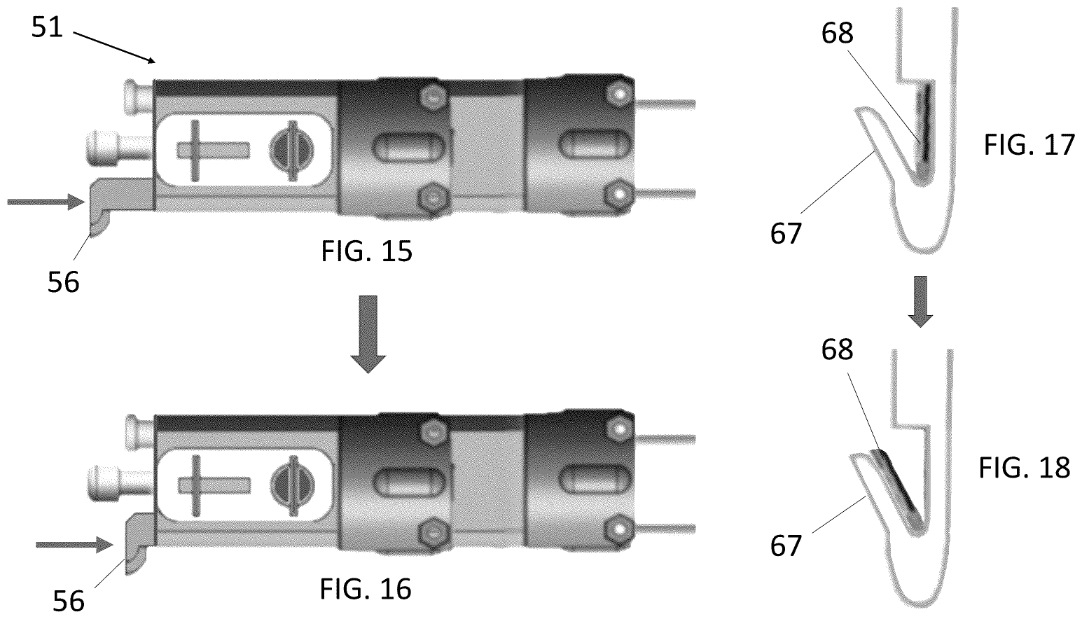

Referring to FIG. 15-18, pushing the gripper control 56 into the body of the proximal control handle 51, extends the gripper 68 outward and it may or may not directly abuts the grasper arm 67. Pulling the grasper control 56 away from the body of the proximal control handle 51 retracts the gripper 68 away from the grasper arm 67 back to the shaft 71 of grasper end 66. Gripper arm 68 may extend outward at any angle via any type of spring or hinge element connecting it to the grasper end 66. The grasper control 56 may be any length, width, or thickness, and may be any type of shape. The gripper 68 may be any length, width, or thickness, and may take any polygonal, circular, or elliptical shape, and may be composed of any combination of materials outlined above. The gripper 68 may also have one or more protrusions from its surface facing the grasper arm 68. These protrusions may of any shape, such as a barb, spear, hook, rod, or be any portion of any type of polygon, sphere, or ellipsoid, and may be arrayed along the surface of the gripper 68 in any formation or distribution.

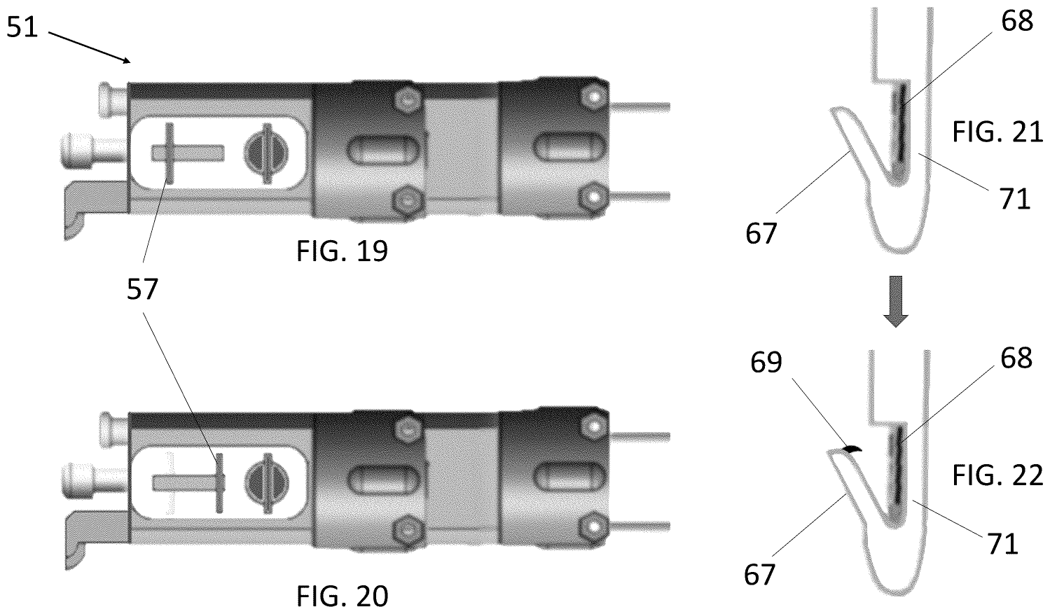

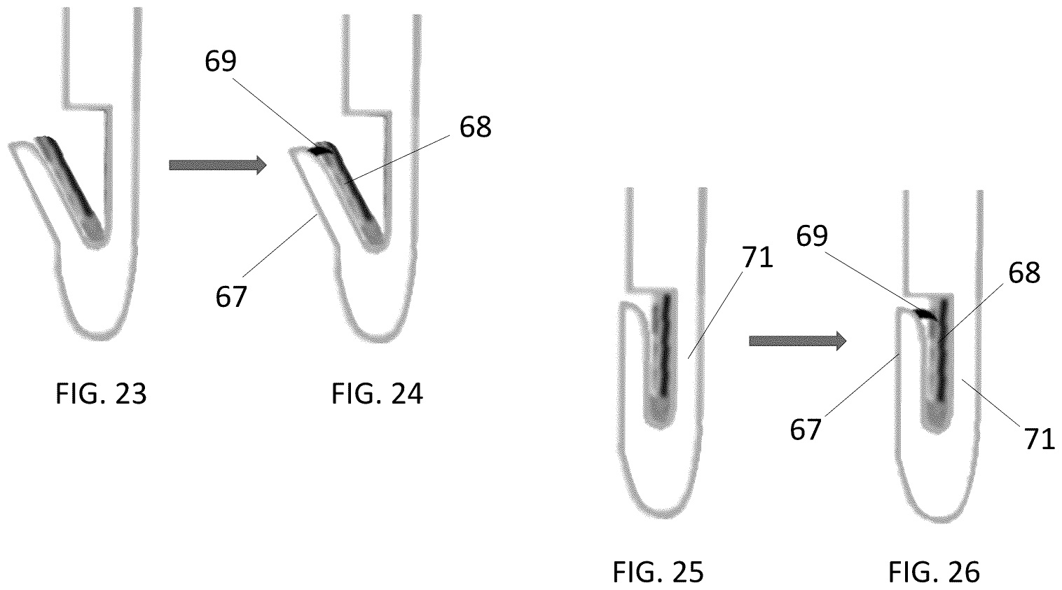

FIG. 19-32 illustrates the form and function of the laceration element 69. As shown in FIG. 19-20 pushing the laceration engagement control 57 forward extends the laceration element 69 outward from it associated member. In FIG. 21-22, the laceration element 69 extends outward from the grasper arm 67. FIG. 21-22 shows element 69 extending outward from grasper arm 67 when the arm 67 is open and the gripper 68 is close to shaft 71, but element 69 may extend from grasper arm 67 when gripper 68 is next to arm 67 (FIG. 23-24), and element 69 may extend from grasper arm 67 when both the grasper arm 67 and gripper 68 are closed next to shaft 71 (FIG. 25-26).

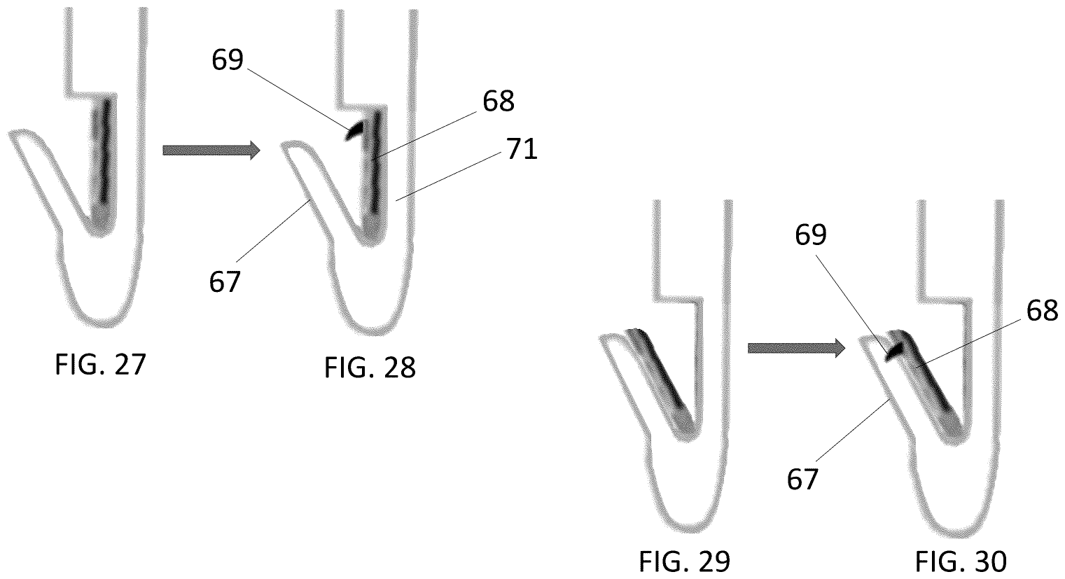

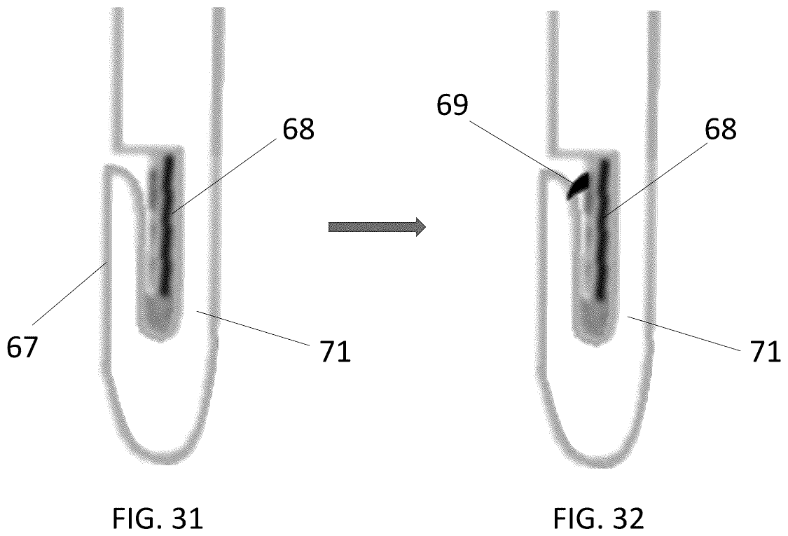

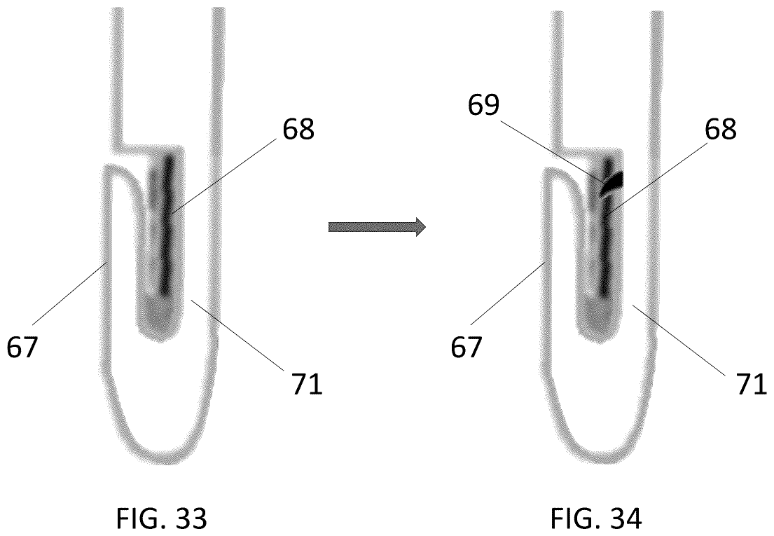

Alternatively, the laceration element 69 may extend from either the gripper 69 or from the shaft 71 of the grasper end 66. The laceration element 69 may be extended from the gripper 68 whether the gripper is next to shaft 71 (FIG. 27-28), whether the gripper is next to grasper arm 67 (FIG. 29-30), or whether the grasper arm and gripper are both closed next to shaft 71 (FIGS. 31-32). FIG. 33-34 shows the laceration element 69 extending directly from the shaft 71.

In another aspect, laceration element 69 may be located anywhere on either the grasper arm 67, gripper 68, or shaft 71. Laceration element 69 may be of any polygonal, circular, ellipsoid, or other shape, of any length, thickness, or width, and may have a sharp tip, and may be composed of any combination of materials outlined above.

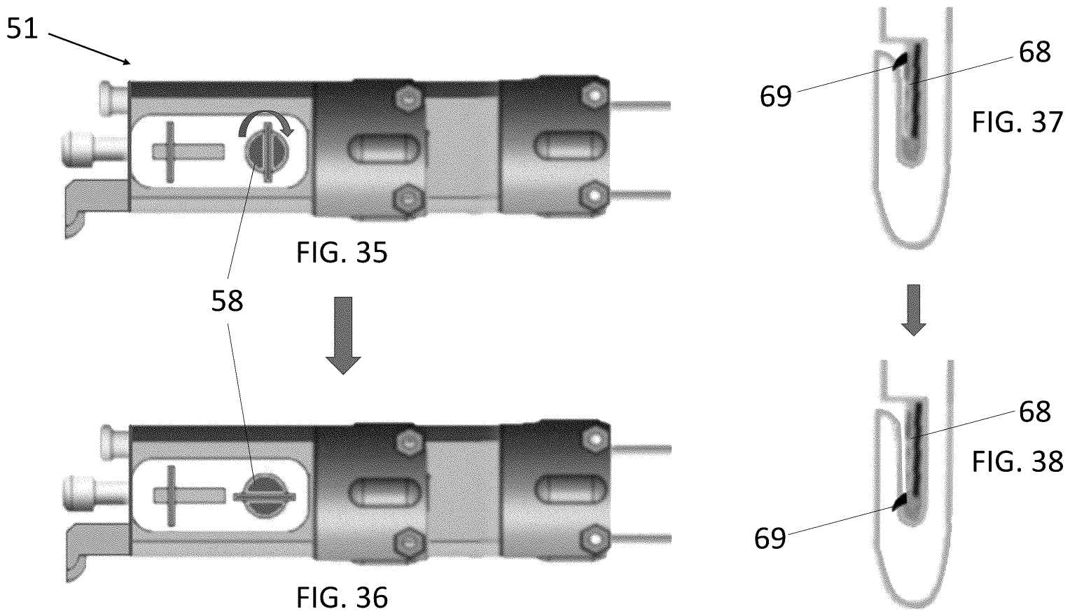

FIG. 35-38 illustrate the actuation of the laceration element. The laceration movement control 58 is rotated, causing the laceration element 69 to move from one end of its associated element to the other end. By example only, the laceration movement control 58 is shown as a rotational knob, but may be any kind of control, including, but not limited to, a control with a push/pull element, a button, or any type of digital control. By example only, the laceration element 69 is shown affixed to the gripper 68, but may be attached to any other element, such as the grasper arm 67, or the shaft 71. By example only, the laceration element 69 is shown moving from top of the gripper 68 to the bottom of the gripper 68, but the laceration element could move from bottom to the top of the gripper 68 or of the grasper arm 67 or shaft 71. Also, the laceration element 69 may move follow any other path, such as, but not limited to, a diagonal, rectangle, zig-zag, circular, elliptical, or other paths.

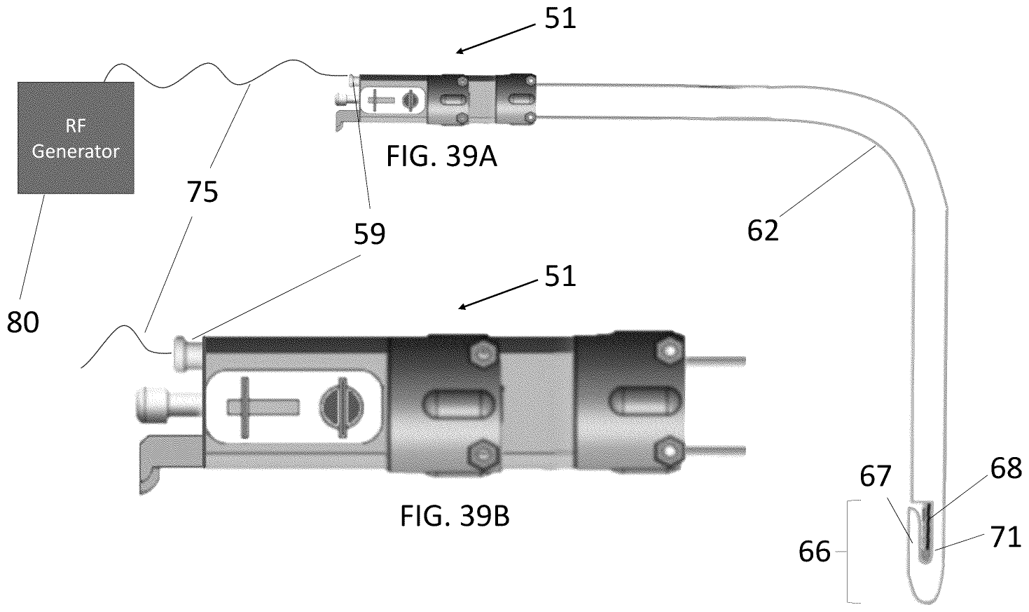

FIG. 39A-B show the set-up and function of an electrosurgical wire with the leaflet grasper. Any type of radiofrequency current generator 80 is connected to an electrosurgical wire 75. Electrosurgical wire 75 enters the proximal control handle 51 via electrosurgical wire port 59. The electrosurgical wire 75 goes through a lumen (not shown) in proximal control handle 51, going through the shaft 62, until the wire runs in either the grasper arm 67, gripper 68, shaft 71, or in any combination. The end of the electrosurgical wire 75 may end in a straight line, diagonal, rectangle, zig-zag, circular, elliptical, or other paths. Activation of the wire 75 by the radiofrequency current generator 80 may score or fully-thickness cut any leaflet tissue captured in the leaflet grasper end 66.

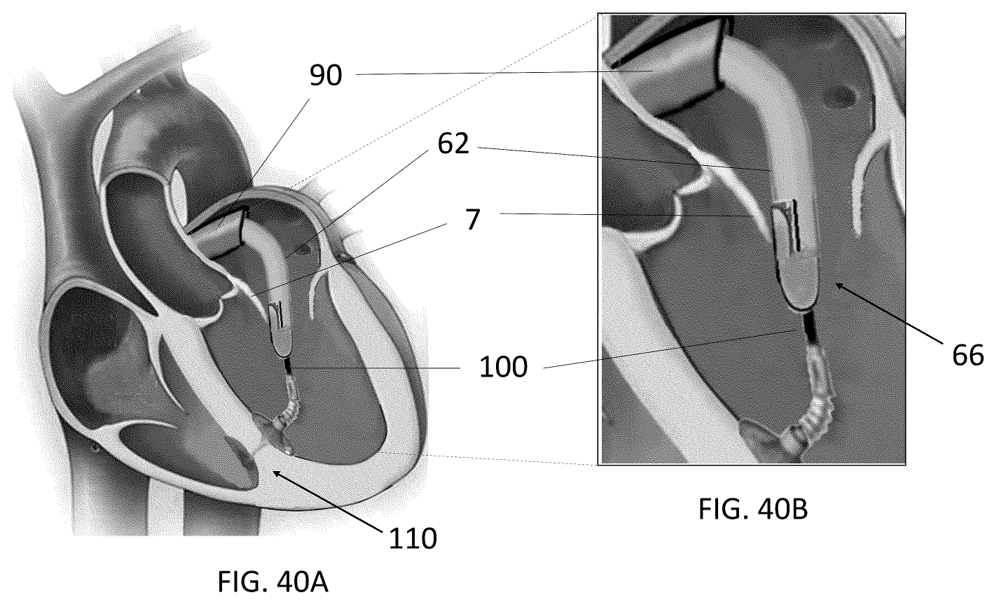

Cutting Mitral Leaflet Tissue with Leaflet Grasper [FIG. 40A-51B]

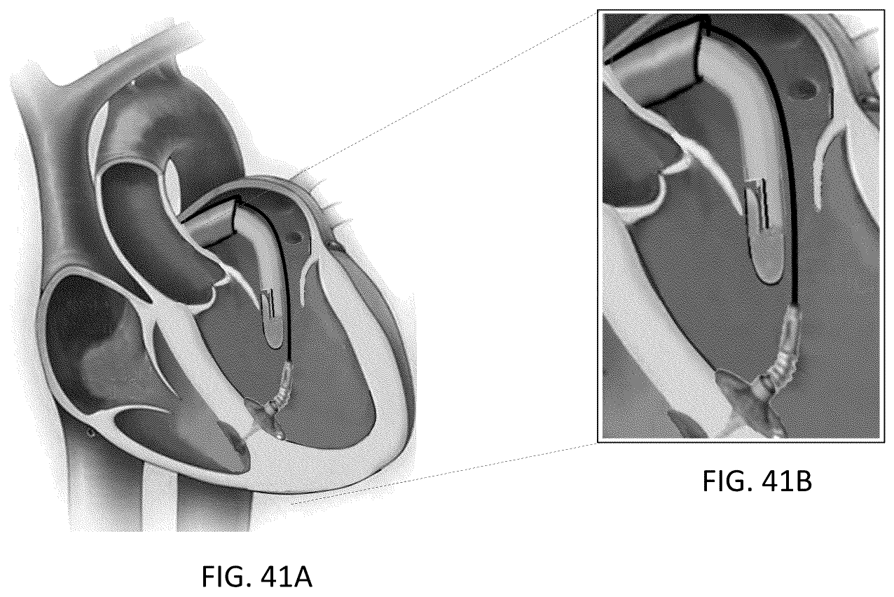

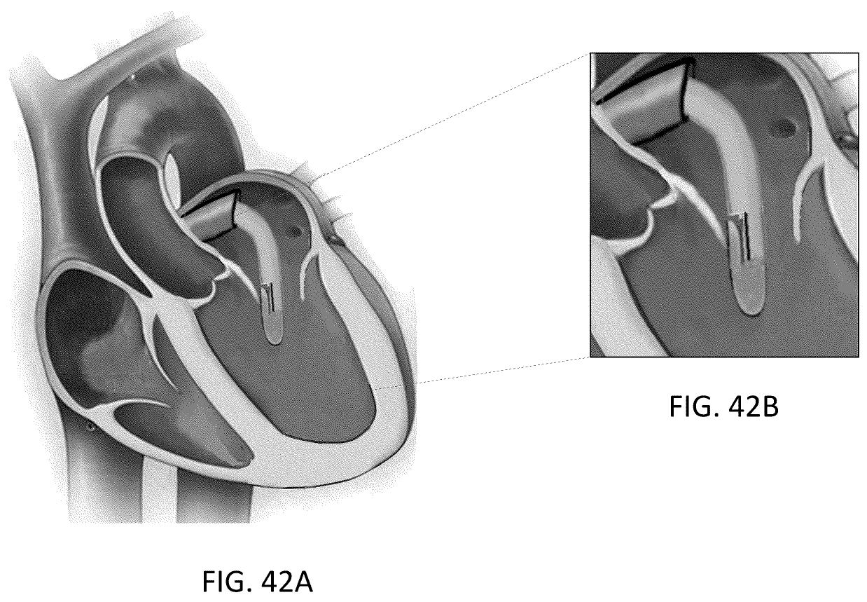

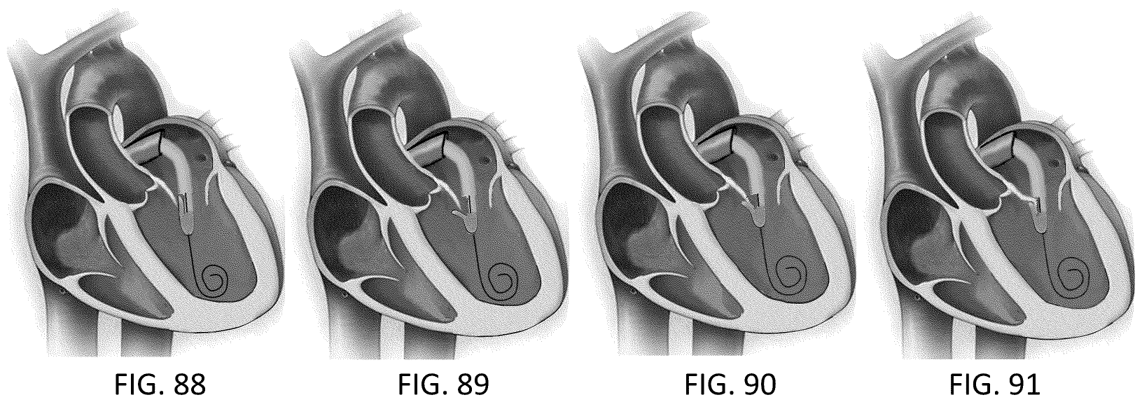

FIG. 40A to 42B illustrate the methods of advancing the leaflet grasper end 66 next to anterior mitral leaflet tissue 7 of the mitral valve. In FIGS. 40A-B, via trans-septal sheath 90, the leaflet grasper end 66 of leaflet grasper 50 is advanced over guidewire 100, which is connected to inter-septal anchor 110. FIG. 88 shows the leaflet grasper 50 advancing over guidewire 100 without inter-septal anchor 110. Guidewire 100 may be of any diameter or material and enters a lumen (not shown) of the leaflet grasper 50, exiting the wire port 61 of the proximal control handle 51 of leaflet grasper 50 (FIG. 11B). For example only, the entry point to the lumen is shown at the end of the leaflet grasper end 66, but the entry point may be along any portion of the leaflet grasper end 66, or may even be anywhere along shaft 62 of the leaflet grasper 50. As the leaflet grasper end 66 is being advanced over guidewire 100, rotation of deflection knob 53 bends shaft 62 (FIG. 12A-B) so that is may follow the path of guidewire 100 without excessive resistance. FIG. 41A-B illustrate the same steps of moving the leaflet grasper end 66 next to anterior mitral leaflet tissue 7, but the leaflet grasper end 66 is not advancing over guidewire 100, but adjacent to it. FIG. 42A-B illustrate the same steps of moving the leaflet grasper end 66 next to anterior mitral leaflet tissue 7, but there is no guidewire 100 or anchor 110.

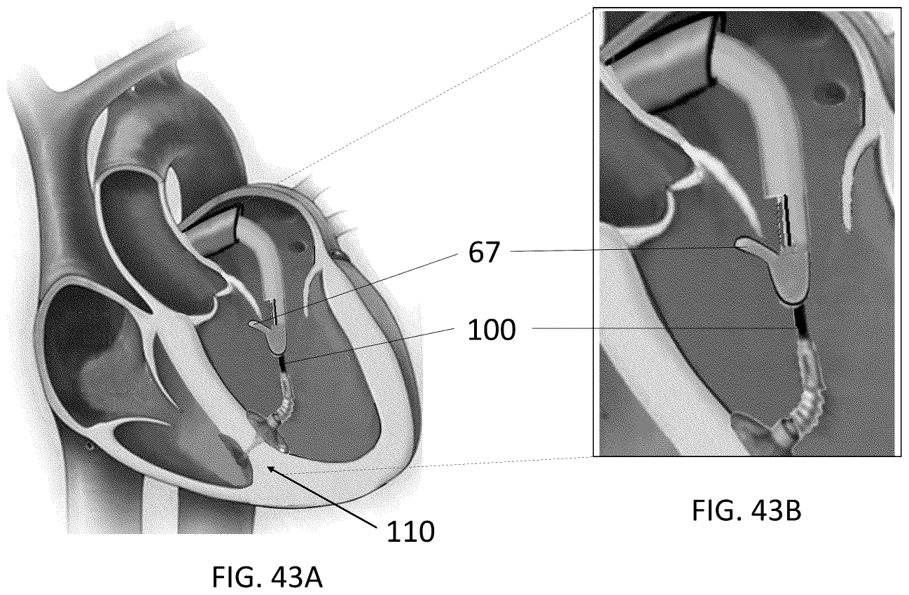

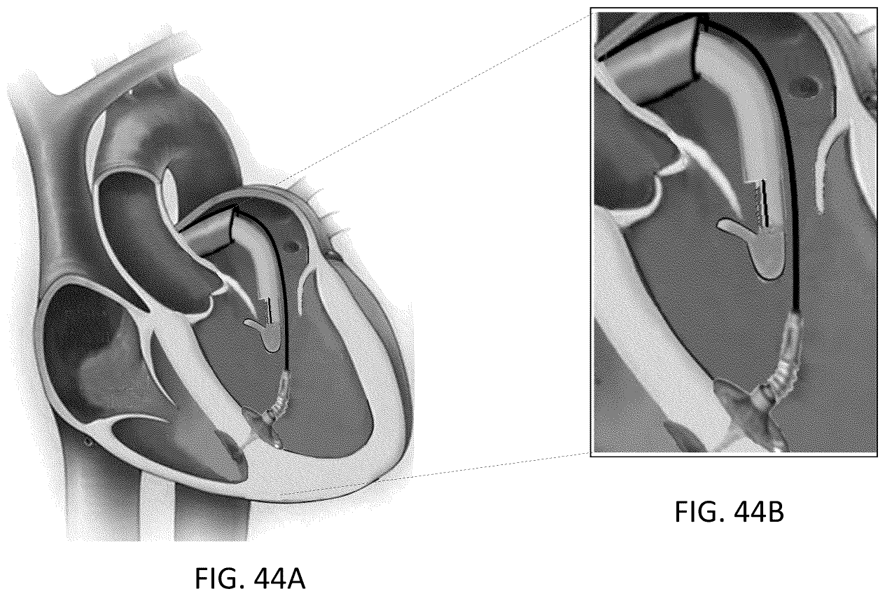

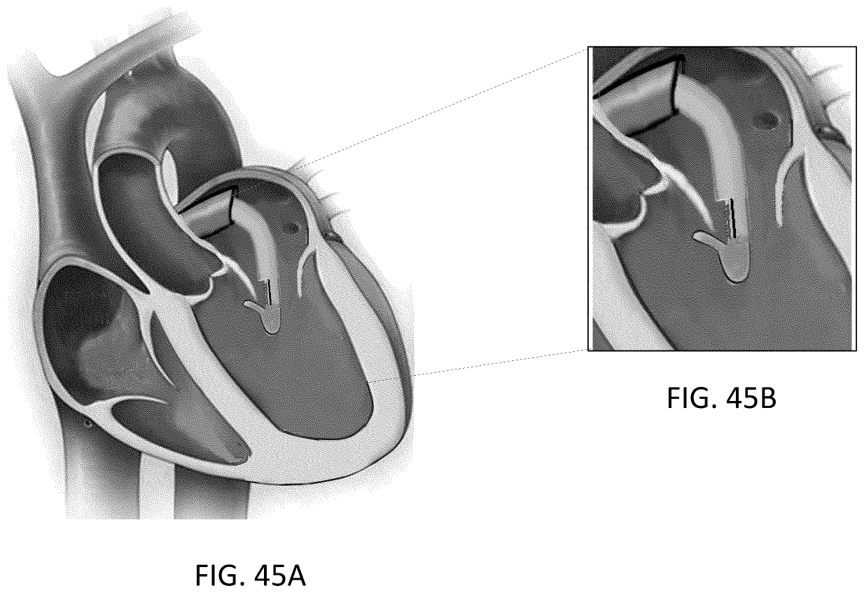

FIG. 43A-45B illustrate the method of opening the leaflet grasper arm 67 of leaflet grasper end 66 to allow capture of anterior mitral leaflet 7. FIG. 43A-B shows the leaflet grasper end 66 advanced over guidewire 100 connected to anchor 110. FIG. 89 shows the leaflet grasper advanced over guidewire 100 not connected to anchor 110. In this position, grasper control 54 of proximal control handle 51 is rotated (FIG. 13-14) until leaflet grasper arm 67 is opened to the desired angle to capture anterior mitral leaflet 7. FIG. 44A-B illustrate this process when grasper end 66 is not over guidewire 100, but adjacent to this, and FIG. 45A-B illustrate this process when there is no guidewire 100 or anchor 110.

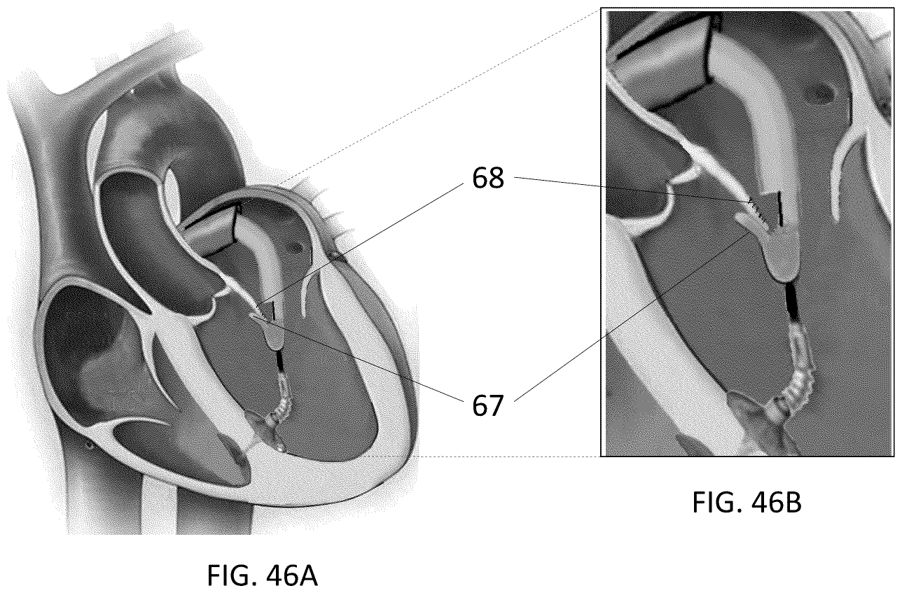

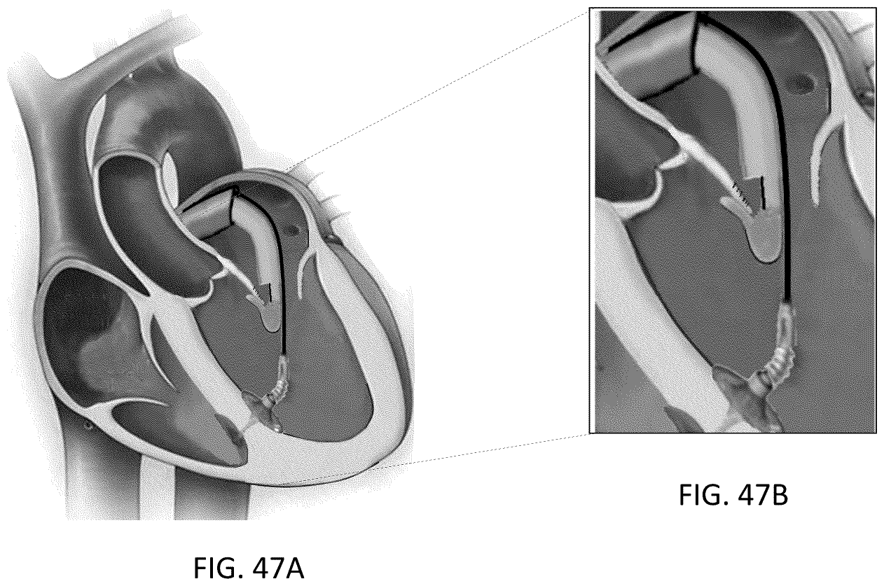

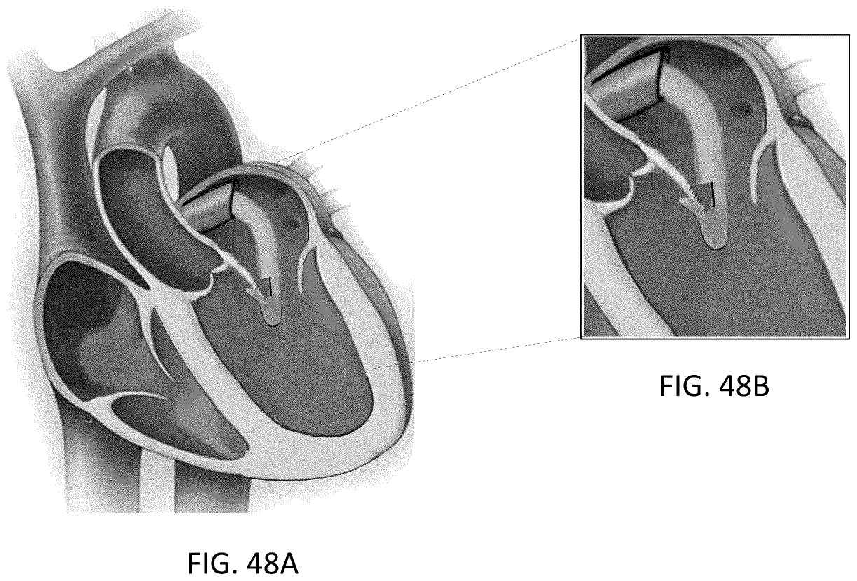

FIG. 46A—48B illustrate the method of grasping the anterior mitral leaflet 7 with gripper 68. FIG. 46A-B show the leaflet grasper end 66 advanced over guidewire 100 connected to anchor 110 with leaflet grasper arm 67 opened and anterior mitral leaflet 7 inside the leaflet grasper arm 67. FIG. 90 shows the leaflet grasper end 66 advanced over guidewire 100 not connected to anchor 110 with leaflet grasper arm 67 opened and anterior mitral leaflet 7 inside the leaflet grasper arm 67. In this position, the grasper control 56 of proximal control handle 51 is advanced (FIG. 15-16), until gripper 68 falls on top of anterior mitral leaflet 7, thereby capturing leaflet 7 between leaflet grasper arm 67 and gripper 68. This capture may or may not be facilitated by one or more additional fixation elements (not shown) attached to gripper 68. FIG. 47A-B illustrate this process when grasper end 66 is adjacent to, but not over, guidewire 100, and FIG. 48A-B illustrate this process when there is no guidewire 100 or anchor 110.

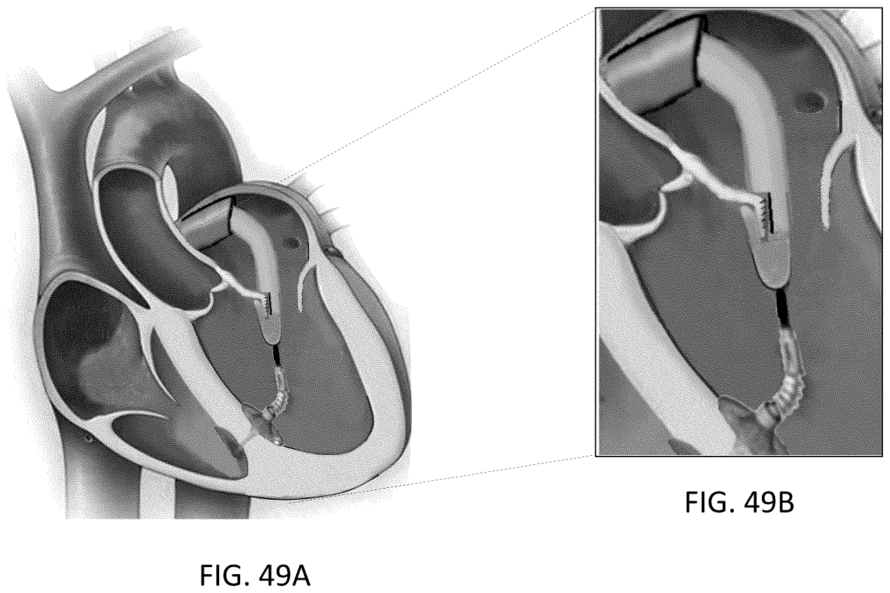

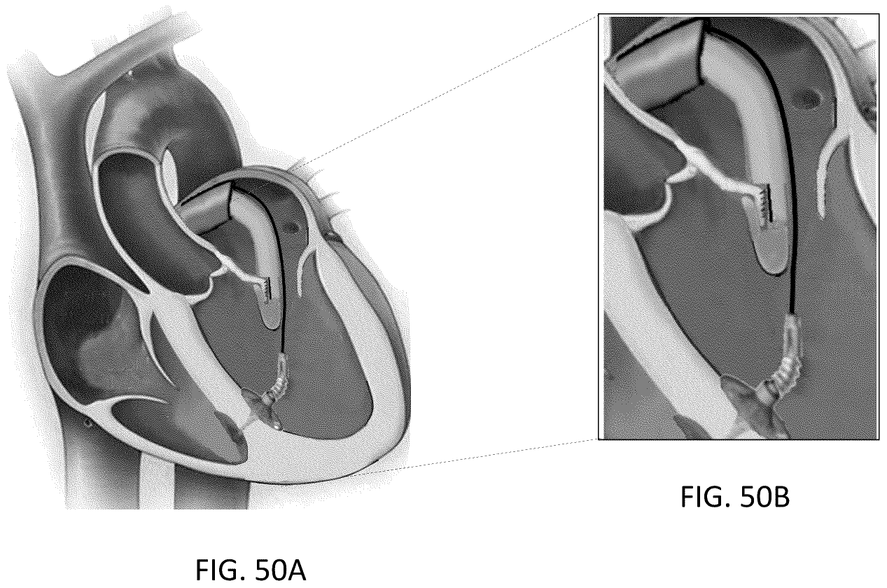

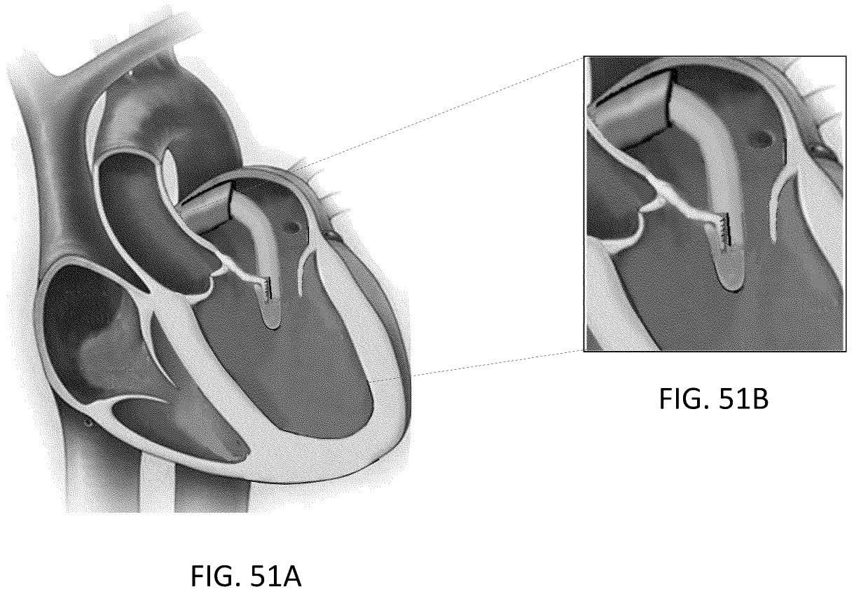

FIG. 49A-51B illustrate the method of closing the leaflet grasper end 66 with captured anterior mitral leaflet 7. FIG. 49A-B show the leaflet grasper end 66 advanced over guidewire 100 connected to anchor 110. FIG. 91 shows the leaflet grasper end 66 advanced over guidewire 100 nt connected to anchor 110. In this position, the grasper control 54 of the proximal control handle 51 is rotated in the direction that allows the leaflet grasper arm 67 and gripper 68 to close against the shaft 71 of the leaflet grasper end 66 (FIG. 14). FIG. 50A-B illustrate this process when grasper end 66 is adjacent to, but not over, guidewire 100, and FIG. 51A-B illustrate this process when there is no guidewire 100 or anchor 110.

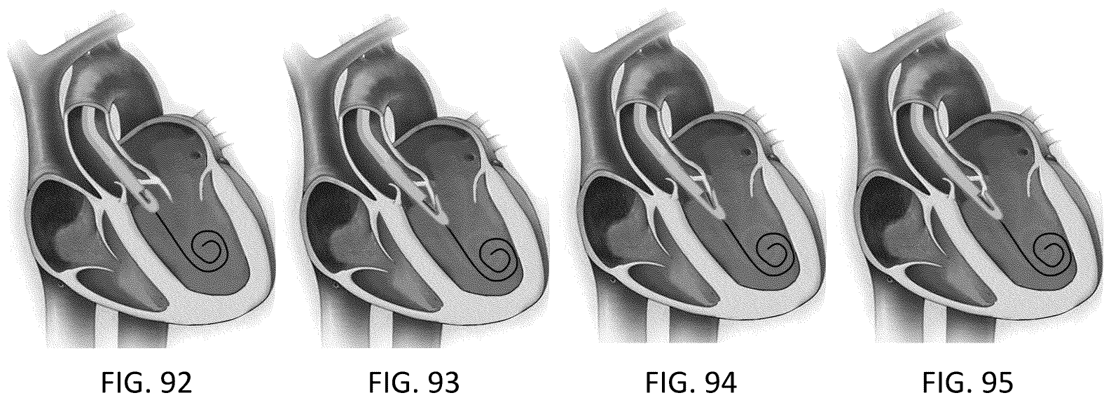

According to another aspect, the steps of advancing leaflet grasper over a wire without an anchor, opening leaflet grasper arm, grasping leaflet, and closing leaflet grasper end, as detailed in [00147], [00148], [00149], and [00150], respectively, can occur with the leaflet grasper end going through aortic valve, as detailed in FIGS. 92-95.

After final step described in [00100] one of more laceration elements [0093-0096] may be used to lacerate the captured tissue. Alternatively, the leaflet may be cut by radiofrequency electrical current per [0097]. Finally, the leaflet grasper arm 67 may be opened and gripper 68 lifted so that the cut leaflet is released, or the grasper end 66 may stay closed and be retracted, thereby extracting capture tissue from the rest of the leaflet.

Grasping and Cutting Aortic Leaflet Tissue with Standard Leaflet Grasper

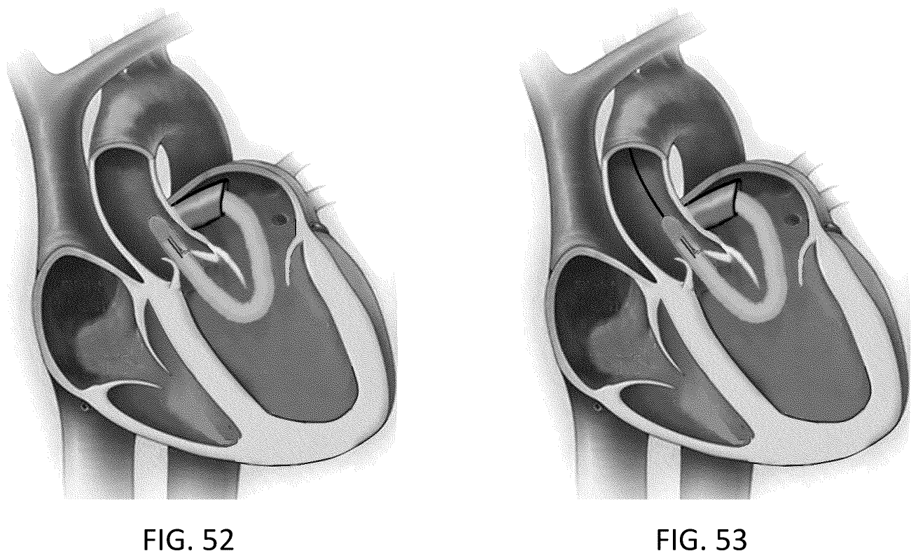

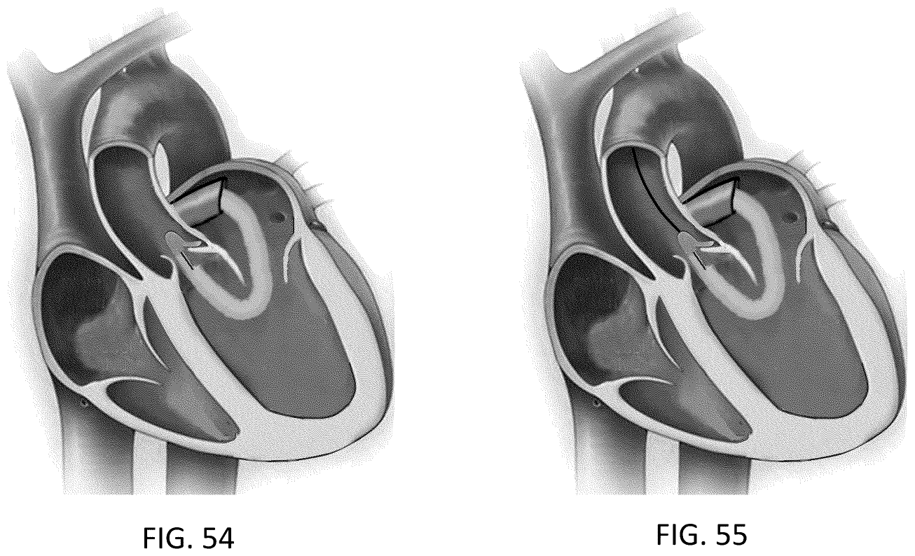

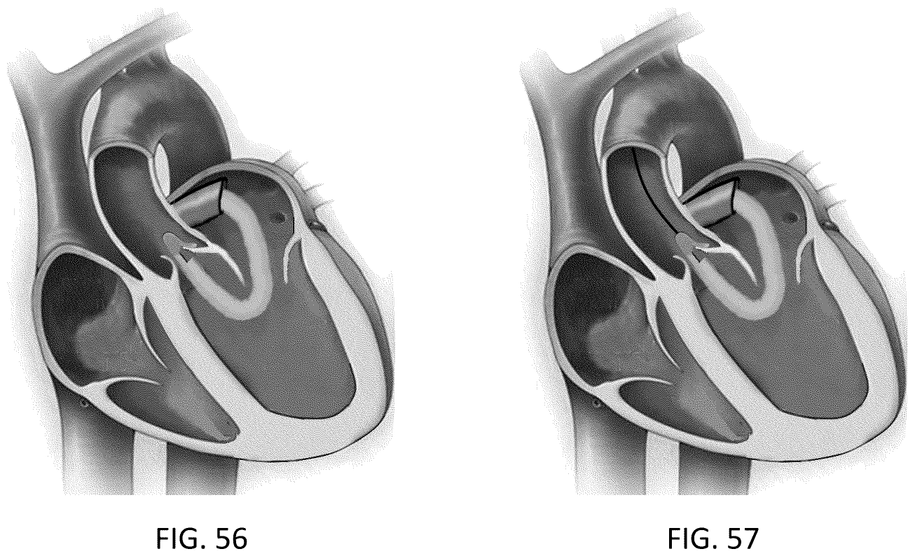

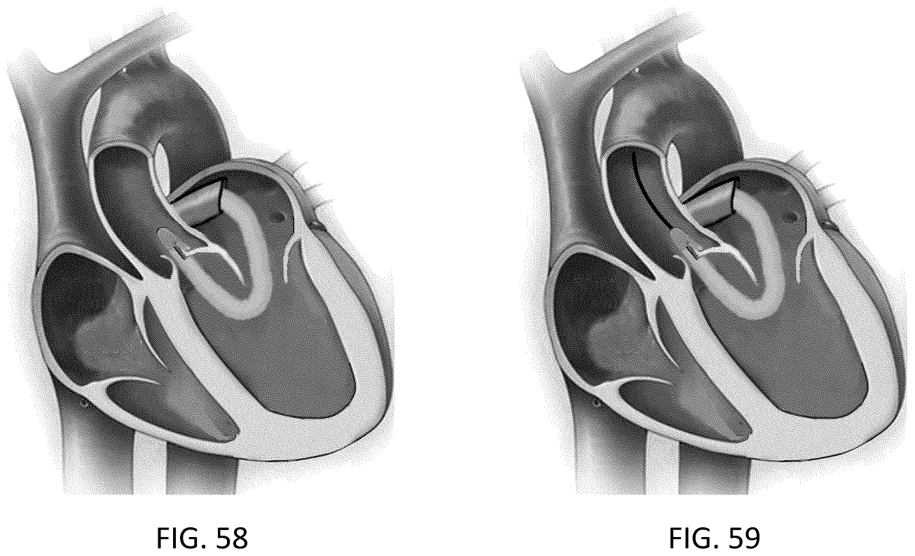

The steps for steering, grasping, and cutting aortic leaflet tissue is the same as the outlined in [00114-00118], just in a different anatomical position. FIG. 52-53 show the leaflet grasper 50 steering with or without a wire from the left atrium 5 into the the left ventricle 9, out of the left ventricular outflow tract 11, across the aortic valve 17. FIG. 54-55 show the process of opening leaflet grasper arm 67 of leaflet grasper end 66 to allow capture of aortic leaflet tissue 17 via steps described above]. FIG. 56-59 illustrate the method of grasping aortic leaflet tissue 17 with gripper 68 and closing the grasper arm via steps described above. Laceration of captured tissue may occur via process described above.

Leaflet Grasper with Alternative Grasper End

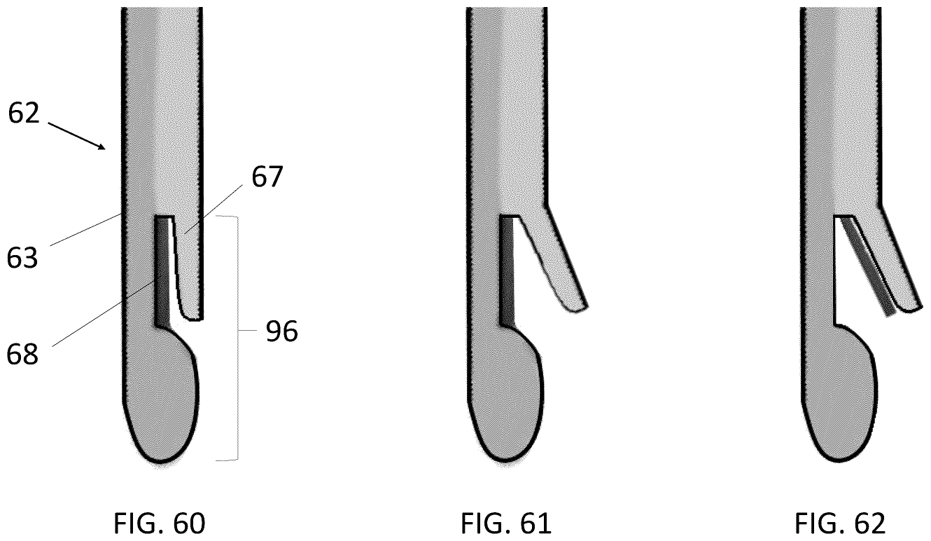

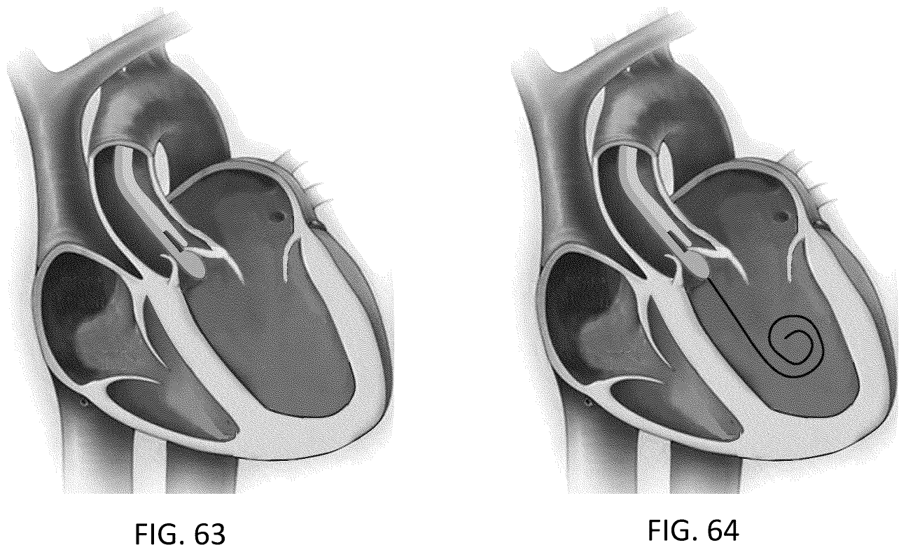

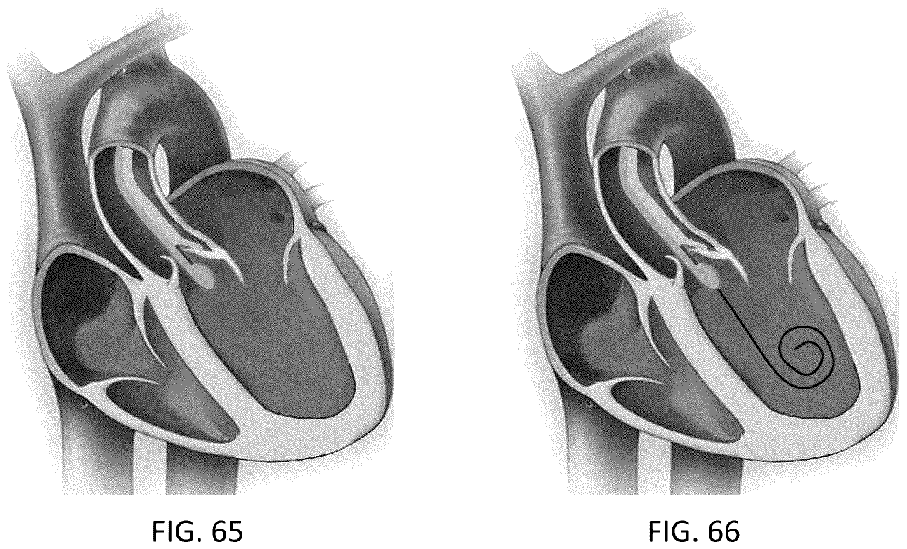

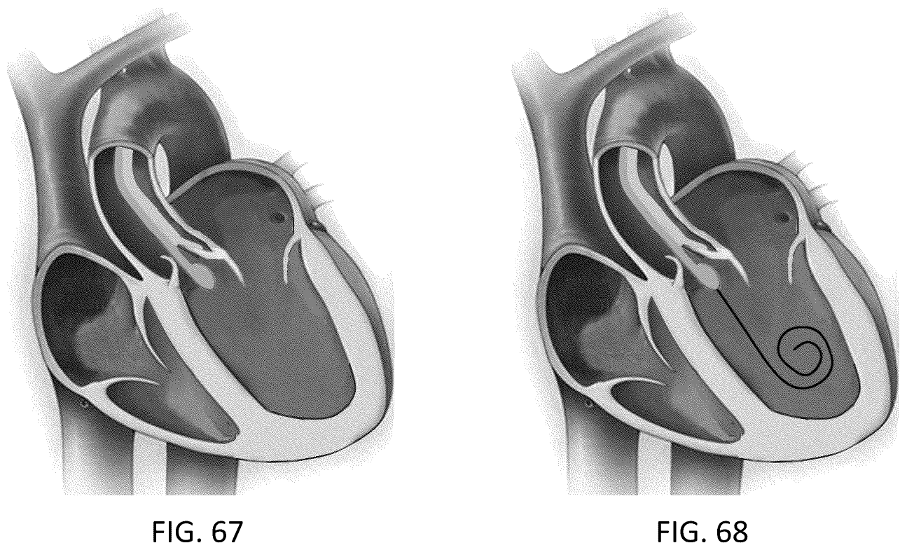

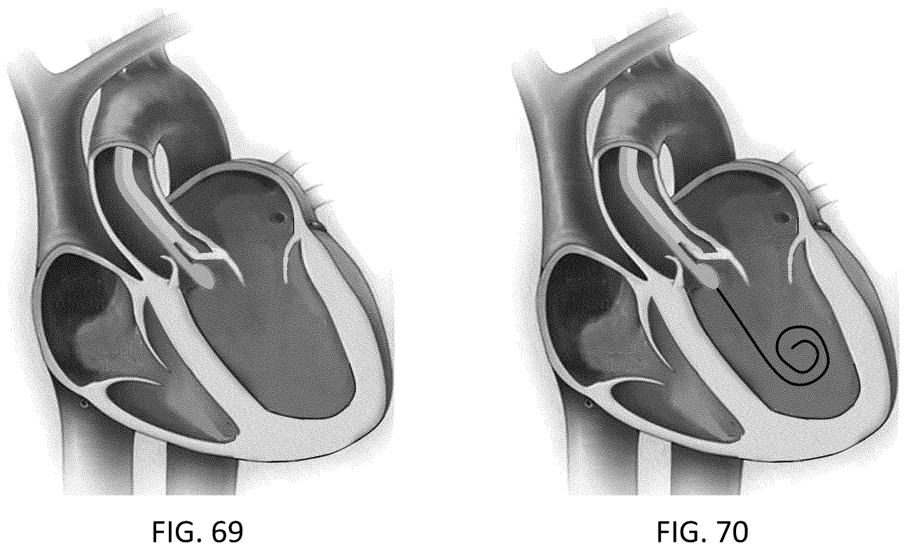

As shown in FIG. 60-62, the leaflet grasper 50 can have a grasper end 96 that differs from grasper end 66. In grasper end 66 the hinge of grasper arm 67 is located in the bottom half of grasper end 66. When grasper arm 67 of grasper end 66 is opened (FIG. 13-14), it allows leaflet tissue to be captured when the tissue is resting above grasper arm 67. In the grasper end 96, the hinge of grasper arm 67 is located in the top half of grasper end 66 closer to the end 63 of shaft 62. In this configuration, when the grasper arm 67 is opened (FIG. 61), it allows the leaflet grasper to capture tissue when the tissue is resting below grasper arm 67. Also, the gripper 68 may close onto leaflet tissue (FIG. 62) when tissue is held by grasper arm 67. Grasping and Cutting Aortic Leaflet Tissue with Leaflet Grasper with Alternative Grasper End FIGS. 63 and 64 show leaflet grasper with grasper end 96 that has advanced up the aorta, across the aortic arch and adjacent to the aortic leaflet without a wire (FIG. 63) or over a wire (FIG. 64). FIG. 65-66 show the process of opening leaflet grasper arm 67 of leaflet grasper 96 to allow capture of aortic leaflet tissue 17 via steps described above]. FIG. 67-70 illustrate the method of grasping aortic leaflet tissue 17 with gripper 68 of grasper end 96 and closing the grasper arm via steps described in above. Laceration of captured tissue may occur via process described herein.

### The Steerable Leaflet Splitter

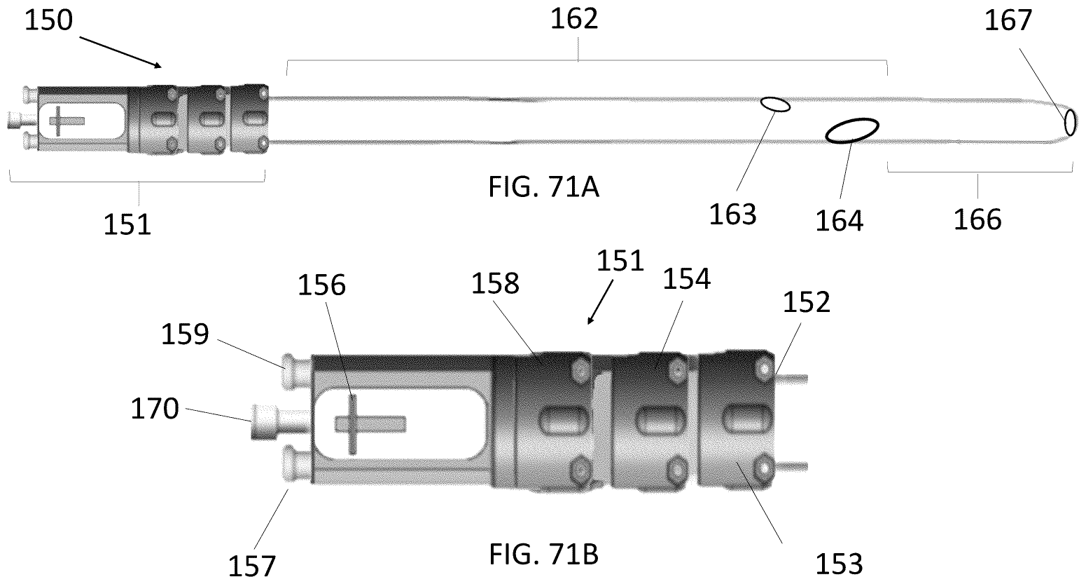

Referring to FIG. 71A-B, the leaflet splitter 150 consists of a proximal control handle 151, catheter shaft 162, and splitter end 166. Any portion of the leaflet grasper may composed of flexible or inflexible materials, including, but not limited to, polyether block amide (PEBA or PEBAX), polyetheretherketone (“PEEK”), polytetrafluoroethylene (“PTFE”), thermoplastic polyurethane (“TPU”), other polymers, stainless steel, cobalt chromium, nickel, titanium, or any combination thereof.

In one aspect, the proximal control handle 151, fused to the proximal portion 152 of the catheter shaft 162, has a deflection knob 153, which controls the deflection of the catheter shaft 162, tip deflection knob 154, which controls deflection of catheter tip 167 of splitter end 166, telescoping catheter deflection knob 158, which controls deflection of telescoping catheter 180, telescoping catheter control 156, which extends or retracts telescoping catheter 180 from telescoping catheter port 164, electrosurgical wire entry port 157, electrosurgical wire exit port 159, and guidewire port 170.

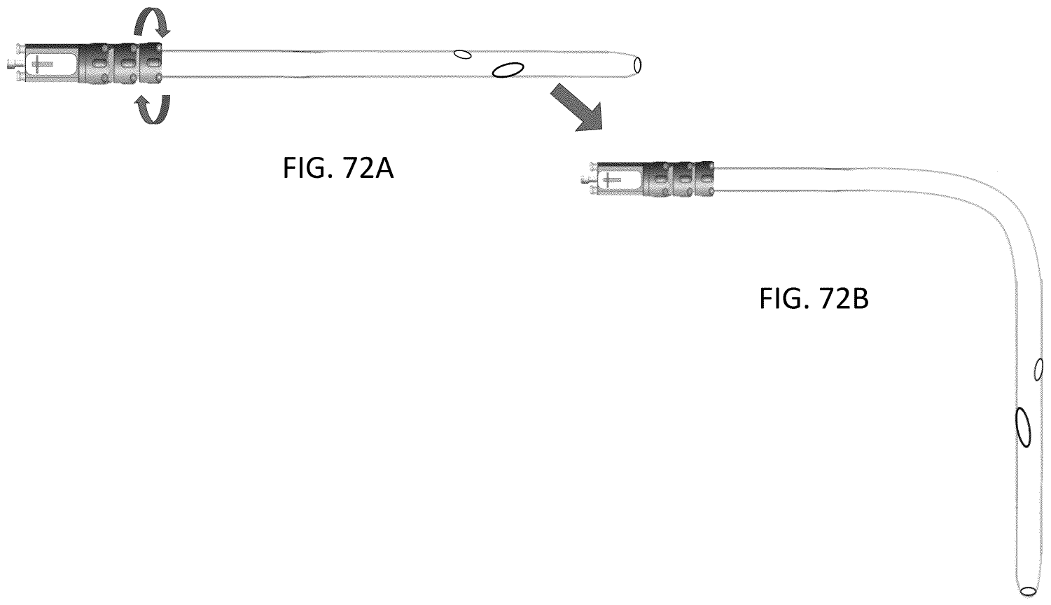

Referring to FIGS. 72A-B, the shaft 162 can be changed from a straight configuration to a bent configuration and back by rotation of the deflection knob 153 of the proximal control handle 151. Rotation of the deflection knob 53 in one direction bends the catheter shaft 162. This bending may be in one or more locations along the length of the shaft, and the one or more locations may bend to any desirable angle so that the shaft may be bent into any type of curve. Rotation of the deflection in the opposite direction reverses the one or more bends along one or more locations of the shaft 162.

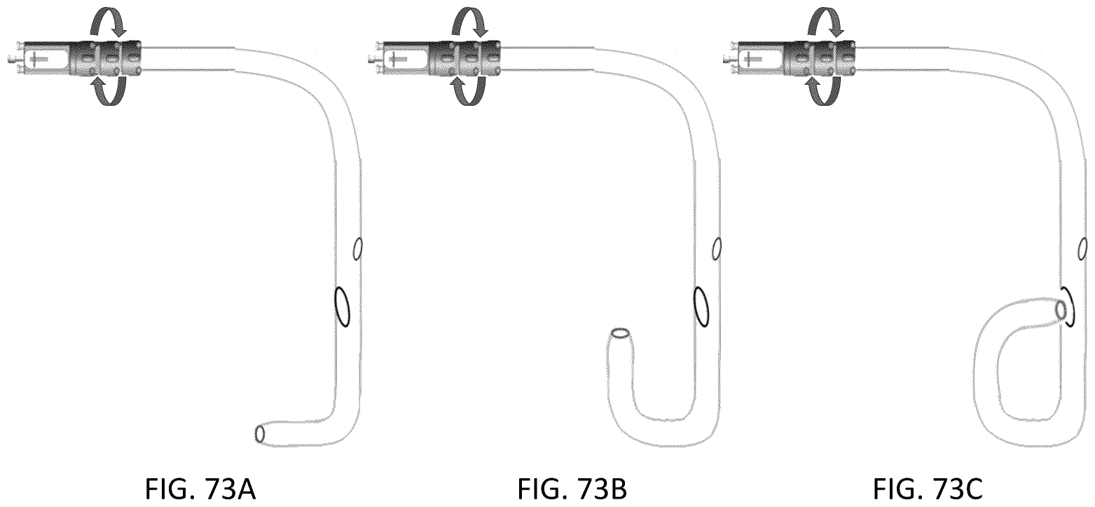

Referring to FIGS. 73A-C, the splitter end 166 can be changed from a straight configuration to a bent configuration and back by rotation of the tip deflection knob 154. Rotation of the tip deflection knob 154 in one direction bends the splitter end 166 any angle so that the catheter tip 167 of the splitter end 166 may make contact with telescoping catheter port 164.

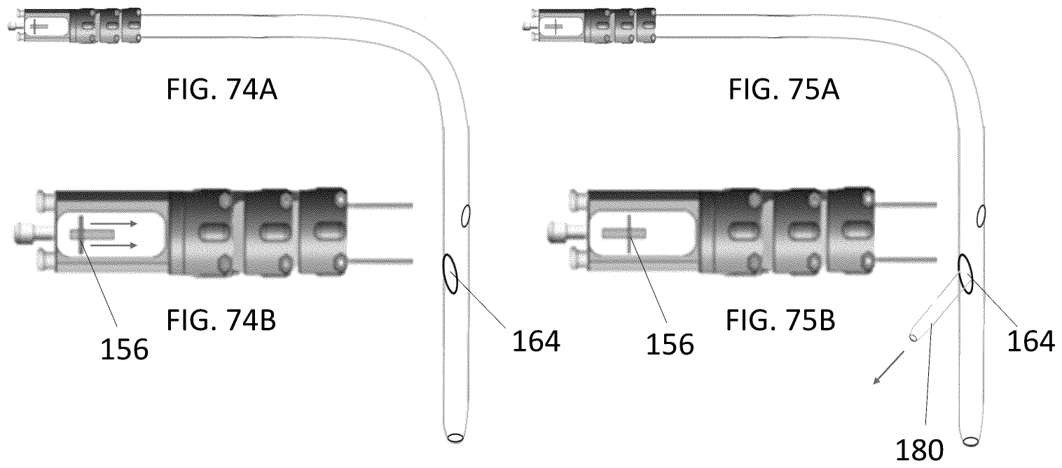

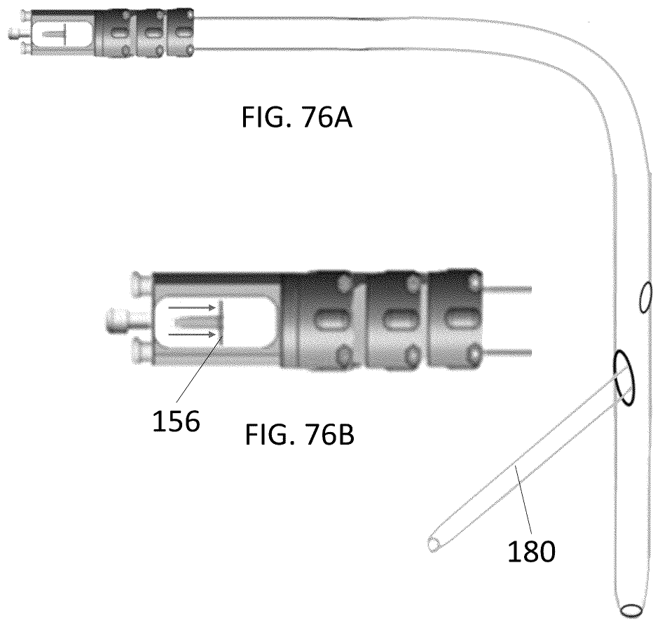

Referring to FIGS. 74-76, advancing the telescoping catheter control 156, advances the telescoping catheter 180 outside the telescoping catheter port 164.

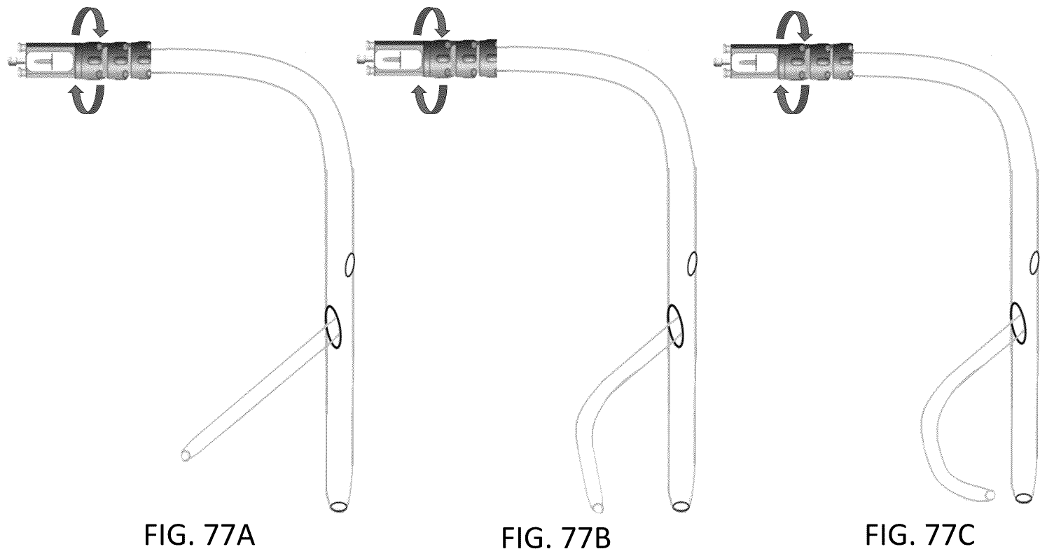

Referring to FIGS. 77A-C, the telescoping catheter 180 can be changed from a straight configuration to a bent configuration and back by rotation of the telescoping catheter deflection knob 158. Rotation of the knob 158 in one direction bends the telescoping catheter to any angle, and reverse rotation of the knob 158 straightens the catheter.

Cutting Mitral Leaflet Tissue with Steerable Leaflet Splitter

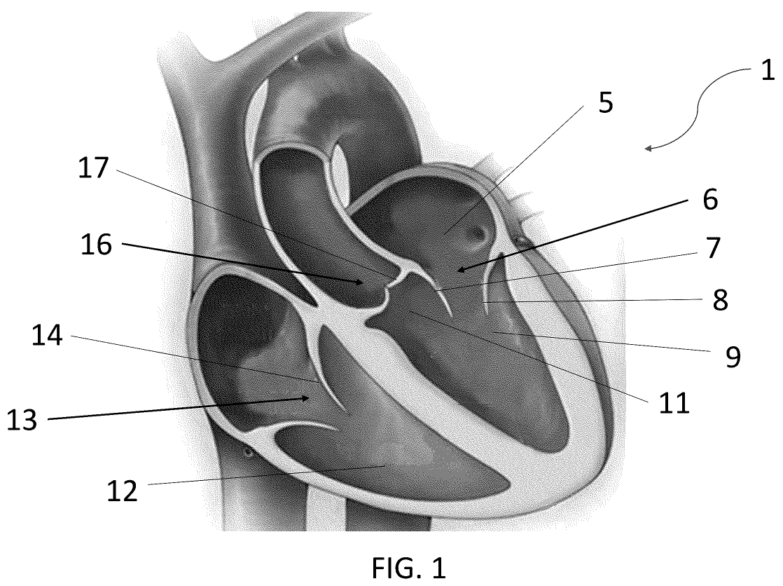

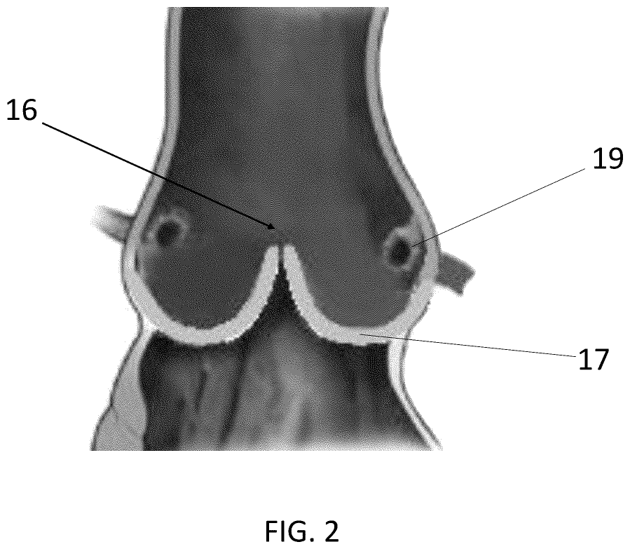

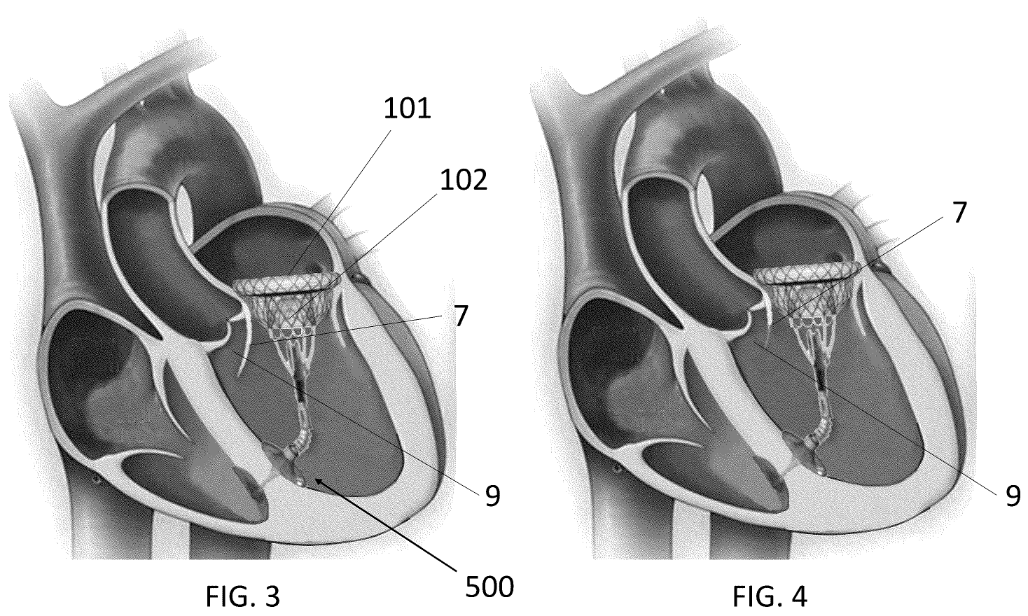

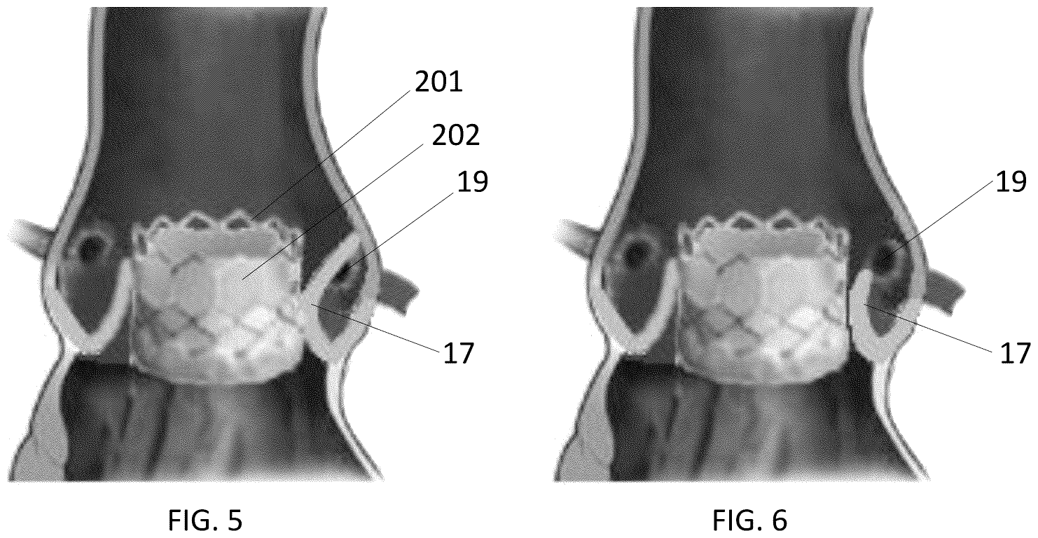

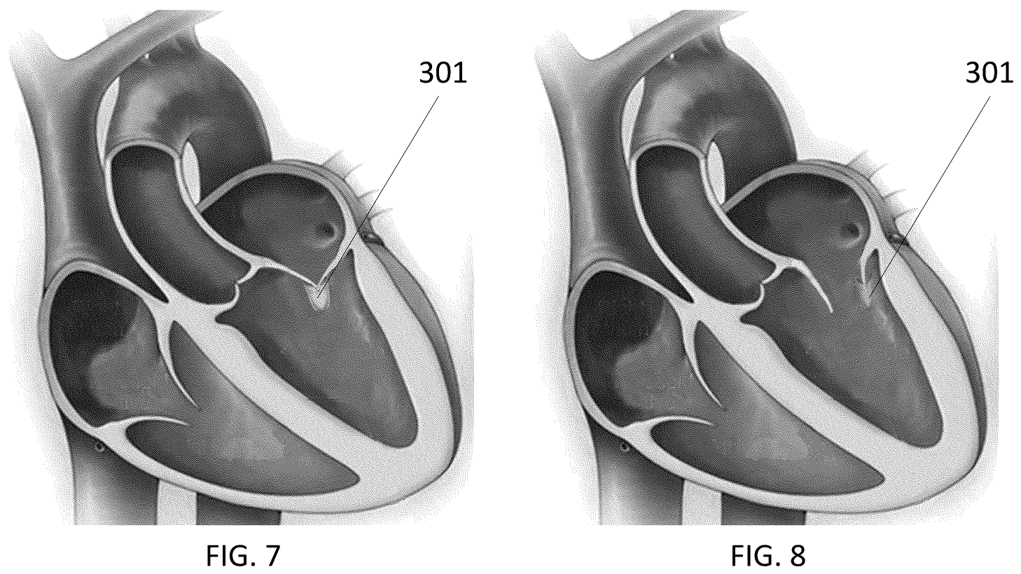

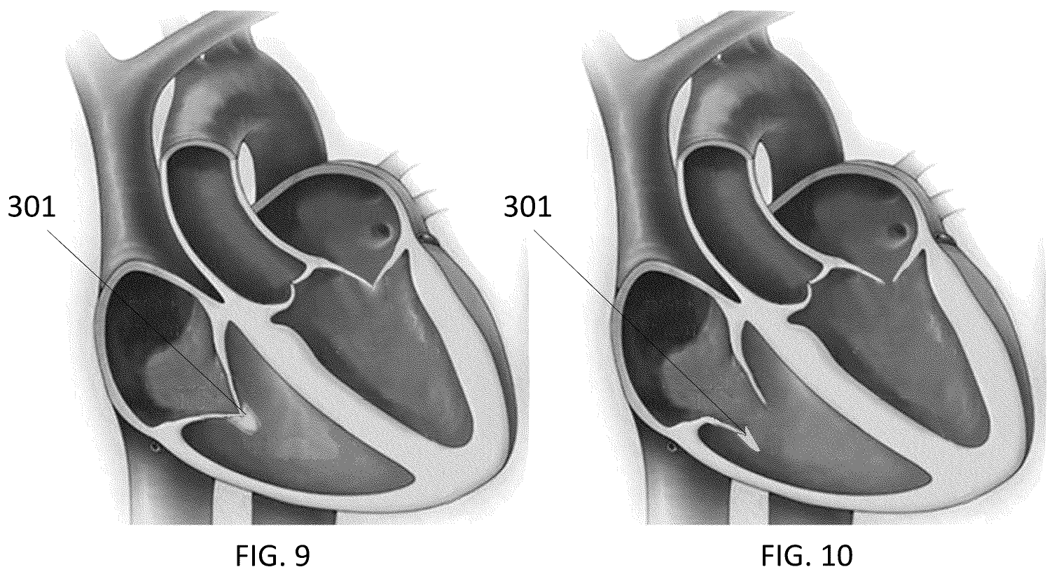

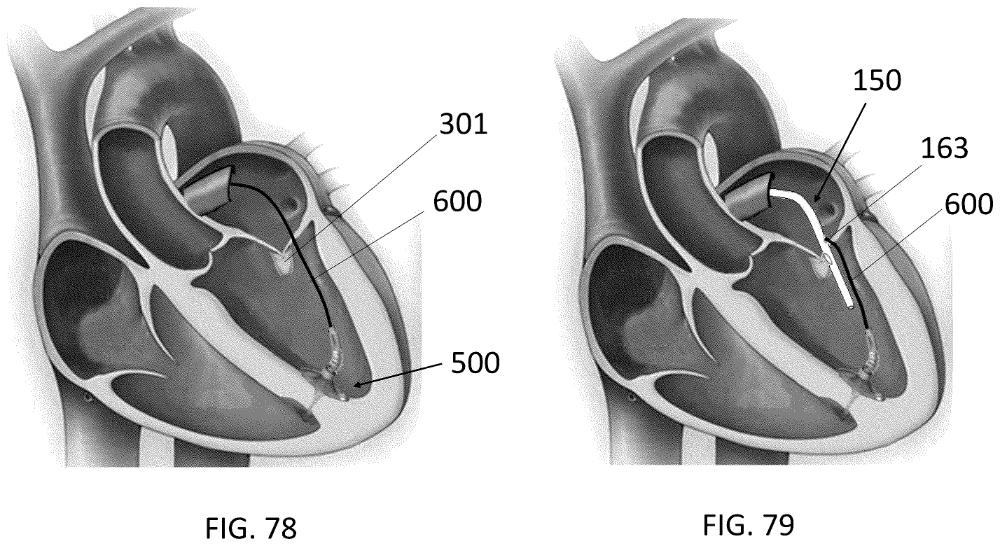

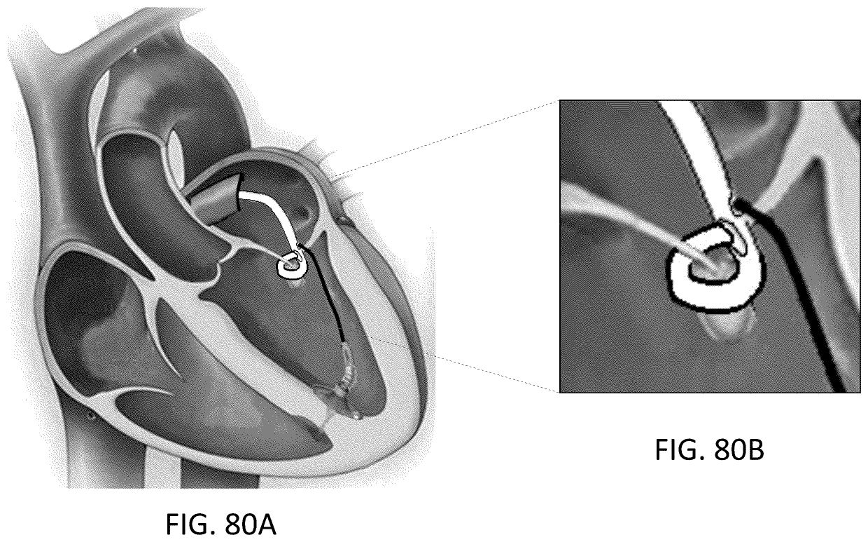

Referring to FIG. 78, a septal anchor 500 with connected guidewire 600 has been placed in a patient with mitral valve repaired with transcatheter edge-to-edge device 301. FIG. 79 shows the leaflet splitter 150 advancing over guidewire 600 with the guidewire 600 entering the guidewire lumen 163. Referring to FIGS. 73A-C and 80A-B, rotation of tip deflection knob 154 bends the splitter end 166 until catheter tip 167 wraps around the anterior mitral leaflet 7, and the catheter tip makes contact with telescoping catheter tip port 164. Without limitation, the catheter tip 167 could be directed to wrap around the posterior leaflet 8 or be used to wrap around any leaflet of a tricuspid valve 13 (FIG. 1) that has been repaired with an transcatheter edge-to-edge device 301 (FIG. 9).

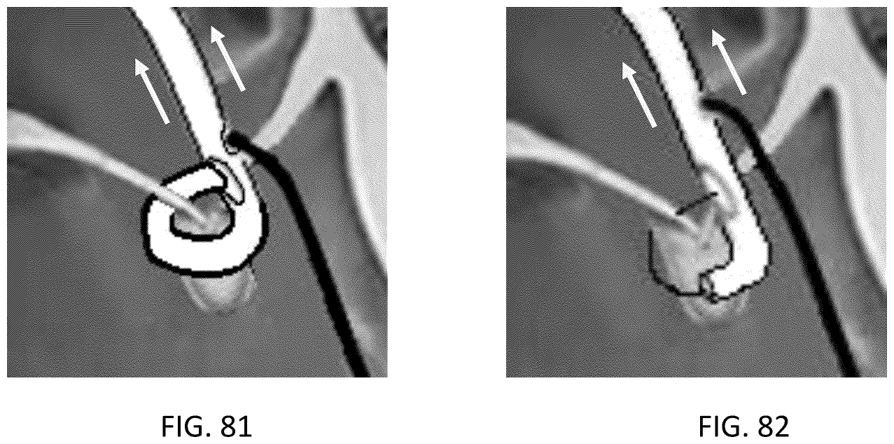

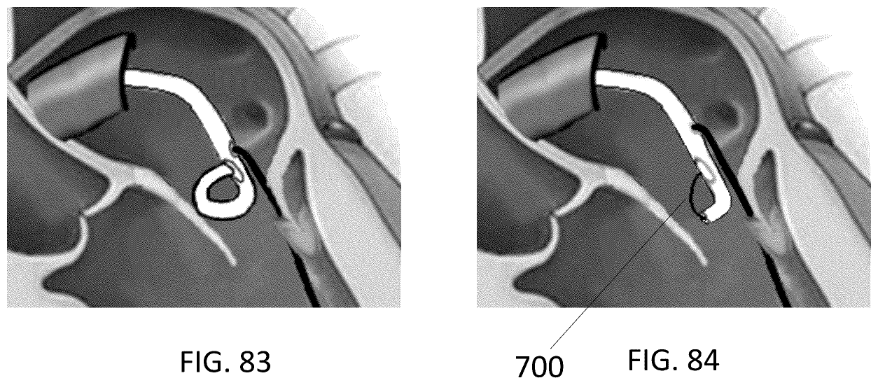

Referring to FIG. 81, the inner edge of the leaflet splitter may have a cutting element or electrical radiofrequency wire (not shown). When the leaflet splitter is pulled upwards, the cutting element or electrical radiofrequency wire, when activated, cuts the leaflet away from the transcatheter edge-to-edge device (FIG. 83) In another method, the a radiofrequency wire 700 is passed into electrosurgical wire entry port 157 (FIG. 71B) through catheter tip 167 back into telescoping catheter tip port 164, exiting electrosurgical wire ext port 159. The wire loop across the leaflet is exposed by unflexing and retraction of catheter tip 167 as shown in FIG. 82. When the leaflet splitter is pulled upwards, the exposed electrical radiofrequency wire, when activated, cuts the leaflet away from the transcatheter edge-to-edge device. (FIG. 84)

Cutting Mitral Leaflet Tissue with Steerable Leaflet Splitter and Telescoping Catheter

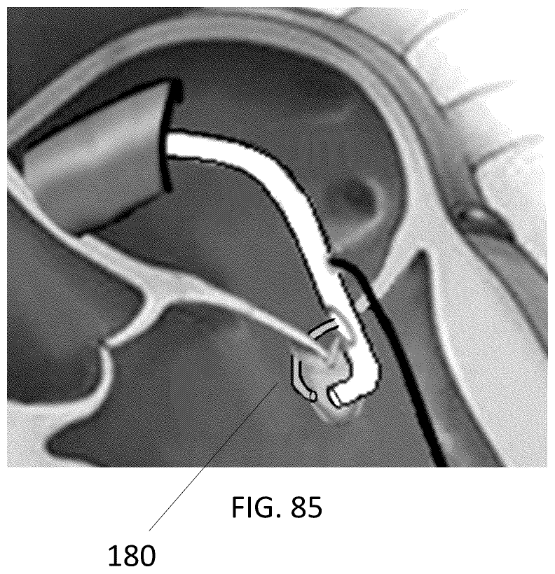

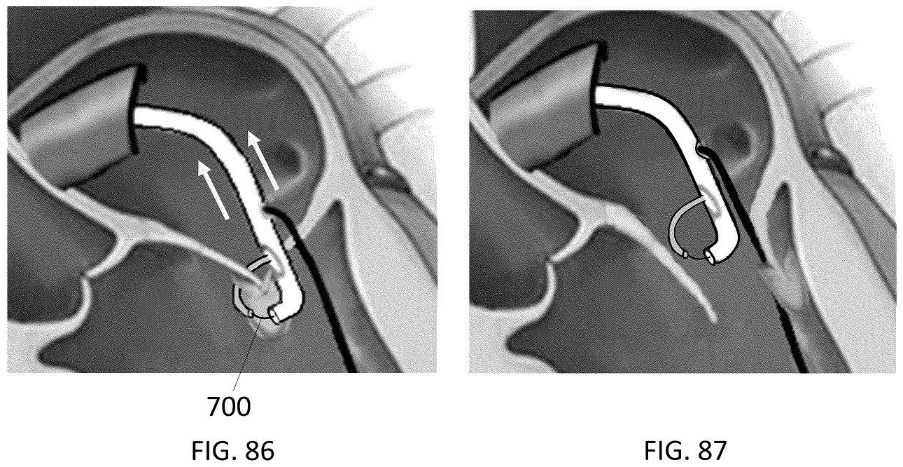

Referring to FIG. 85, the leaflet splitter can be used by encircle and cut the leaflet using the telescoping catheter 180. In this method, after the leaflet splitter 150 is advanced over guidewire 600, rotation of the tip deflection knob 154 bends the splitter end 166. The telescoping catheter 180 is advanced out of the telescoping catheter port 164 by the telescoping catheter control 156 and bent around the leaflet tissue and towards the catheter tip 167 using the telescoping catheter deflection knob 158 (FIGS. 77A-C). In this position an radiofrequency wire may be passed from either the telescoping catheter 180 into the catheter tip 167, or from the catheter tip 167 into telescoping catheter 180. Alternatively, a wire snare (not shown) may be passed into either telescoping catheter 180 or into catheter tip 167 and be used to snare a radiofrequency wire from the opposition catheter. As described above, an exposed wire loop can be pulled upwards to cut the leaflet away from the transcatheter edge-to-edge device (FIG. 86-87)

1. A minimally invasive leaflet grasper for intracardiac implantation into a heart chamber for grasping a heart leaflet and modifying the leaflet, said leaflet grasper comprising:
a proximal end and a distal end and including a control handle on said leaflet grasper proximal end, a grasping end on said leaflet distal end, a catheter shaft extending between said proximal end and said distal end;
said proximal control handle includes a laceration movement control and a grasper control;
said grasping end includes a grasping end main shaft and a grasping arm wherein said grasping arm and said grasping arm main shaft define a leaflet receiving receptacle therebetween and wherein said grasping arm moves from a first position adjacent said grasping arm main shaft to a second position wherein in said second position, said grasping arm extends away from said grasping arm main shaft; and
said grasping end further includes a laceration member on said grasping arm for modifying the leaflet within said cavity; and said laceration element is operatively connected to said laceration movement control and said grasper arm is operatively connected to said grasper control.

2. The minimally invasive leaflet grasper according to claim 1 wherein said proximal control handle further includes a gripper control and said grasping end further comprises a gripper operatively connection to said gripper control wherein said gripper extents within said leaflet receiving cavity.

3. The minimally invasive leaflet grasper according to claim 1 wherein said catheter shaft is steerable and said proximal control handle includes a deflection knob operatively connected to said catheter shaft for altering an orientation of said catheter shaft.

4. A minimally invasive leaflet splitter for intracardiac implantation into a heart chamber for grasping a heart leaflet and modifying the leaflet, said leaflet splitter comprising:
a proximal end, a distal end and a catheter shaft extending therebetween;
a control handle on said leaflet splitter proximal end having a deflection knob operatively connected to said catheter shaft and wherein said catheter shaft is deflectable and said control handle controls deflection of said catheter shaft and wherein said catheter shaft defines at least one port configured for receipt of a guidewire;
a splitter end on said leaf splitter distal end wherein said splitter end is operatively connected to said control handle which controls deflection of said splitter end and wherein said splitter end and said catheter shaft define a center lumen and wherein said splitter end is configured to wrap around itself to encircle the heart leaflet; and
a wire extending within said center lumen for encircling the leaflet.

5. The minimally invasive leaflet splitter according to claim 1 wherein said wire is a radiofrequency energized wire.

6. A method of utilizing a leaflet grasper for intracardiac implantation into a heart chamber for grasping and modifying a heart leaflet comprising the steps of:
inserting the leaflet grasper into a heart chamber wherein said leaflet grasper includes a proximal end and a distal end and including a control handle on said leaflet grasper proximal end, a grasping end on said leaflet distal end, and a catheter shaft extending between said proximal end and said distal end, said grasping end includes a grasping end main shaft and a grasping arm wherein said grasping arm and said grasping arm main shaft define a leaflet receiving receptacle therebetween and wherein said grasping arm moves from a first position to a second position wherein in said second position, said grasping arm extends away from said grasping arm main shaft, and said grasping end further includes a laceration member on said grasping arm for modifying the leaflet within said cavity;
positioning said grasping end adjacent the heart leaflet to be modified, opening said grasping arm to said second position to receive the leaflet within said receptacle;
closing said grasping arm to said first position to secure the leaflet; and
activating the laceration member to modify the leaflet.
